(19) United States
(12) Patent Application Publication
(10) Pub. No.: US 2010/0172991 A1
(43) Pub. Date: Jul. 8, 2010

(54) Extended Release Formulation and Methods of Treating Adrenergic Dysregulation

(76) Inventors: Henry Joseph Horacek, Charlotte, NC (US); Min Michael He, Ellicott City, MD (US); Moise A. Khayrallah, Morrisville, NC (US)

Correspondence Address: MYERS BIGEL SIBLEY & SAJOVEC, PO BOX 37428, RALEIGH, NC, 27627, US

(21) Appl. No.: 12/645,772

(22) Filed: Dec. 23, 2009

Related U.S. Application Data
(63) Continuation-in-part of application No. 12/134,333, filed on Jun. 6, 2008.
(60) Provisional application No. 60/942,934, filed on Jun. 8, 2007.

(51) Int. Cl. A61K 9/14; A61K 31/4168; C07D 233/50; A61P 9/12; A61P 9/04; A61P 25/06; A61P 25/02; A61P 25/30; A61P 3/04; A61P 25/18

(52) U.S. Cl. 424/488; 514/398; 548/333.1

(57) ABSTRACT

A composition and method of treating adrenergic dysregulation by administering the composition is disclosed, wherein the composition comprises a α₂-adrenergic receptor agonist; a pharmaceutically acceptable hydrophilic matrix and a release-retardant of a metal alkyl sulfate. In embodiments, the composition provides a sustained release of the agonist, wherein after administration of the composition no more than once about every 12 hours (e.g., no more than once about every 24 hours) to a subject having a steady state plasma concentration of the α₂-adrenergic receptor agonist, the agonist's plasma concentration peak-to-trough ratio is no greater than about 1.9.

This application is a continuation-in-part of U.S. application Ser. No. 12/134,333, filed Jun. 6, 2008, which claims the benefit of U.S. Provisional Application No. 60/942,934, filed Jun. 8, 2007. The entire contents of these applications are hereby incorporated herein by reference.

### FIELD OF THE INVENTION

Compositions and methods of treatment and prevention of adrenergic dysregulation are disclosed.

### BACKGROUND OF THE INVENTION

Adrenergic dysregulation (hyperadrenergia or hypoadrenergia) refers to abnormal neuronal activation or secretion of the hormone adrenaline and/or the neurotransmitter noradrenaline. Adrenergic dysregulation may occur at both baseline levels of stimulation and in response to external stress. Excessive adrenergic stimulation results in symptoms such as high blood pressure, hyperactivity, physical aggression, motor tics, and insomnia.

Effective drug therapies require control of blood serum levels of drug. Time release hydrophilic matrices are known in the field of drug formulations. For example, one such hydrophilic matrix is hydroxypropyl methylcellulose (HPMC). HPMC matrixes in a surrounding medium of low ionic strength with electrolytes typically hydrate to produce an intact gel layer. An intact gel layer may provide predictable release of an incorporated drug from the matrix by migration through the gel layer. However, at intermediate ionic strengths, the same matrix may lose shape and disintegrate rapidly. Thus, electrolytes present in the surrounding medium may modify the release profile of drugs from HPMC matrices. Modification of the release profile of a drug resulting from differences in matrix environment may be detrimental to the therapeutic usefulness of a drug.

Drugs themselves may also influence the rate of hydration and the rate of gelation of hydrophilic matrixes. (Mitchell, et al., Int. J. Pharm. (1993) 100: 165-173). Therefore, the incorporation of drugs in hydrophilic matrices may result in unpredictable dissolution profiles, which may result in unpredictable therapeutic efficiency of the dosage forms.

Drug release from an oral solid extended release dosage form and subsequent absorption of the drug from the gastro-intestinal tract into the blood stream is dissolution-rate dependent and may be slow and irregular especially in the case of sparingly water soluble, slightly water soluble, very slightly water soluble, practically water insoluble, or a water insoluble drug, as defined according to the United States Pharmacopeia 24, p 10.

Additives may be added to hydrophilic matrixes to modify the gelling rate and/or the release rate of an incorporated drug. However, the nature of the interaction of a particular drug with the matrix and additive is not generally predictable. This is particularly problematic for drugs administered in low dosages or drugs with limited solubility. It is also difficult to correlate the release rate of a drug with its serum or blood concentration when complex matrix/additive systems are used.

The traditional oral dosage formulations of α₂-adrenergic receptor agonists have disadvantages. The release profile of the traditional oral dose is typically a rapid and bolus release followed by rapid and complete absorption. For example, the traditional oral formulation of clonidine has side-effects including sedation about an hour after the given dose, when the patient may become transiently sedated or fall asleep. Because of the rapid absorption of the drug, the half-life of this dosage form of clonidine is essentially the same as the biological half-life of about four to six hours. Thus, in the traditional formulation of clonidine, the therapeutic effect may wear off too soon and possibly be accompanied with rebound hyperarousal. This may occur in the middle of the night causing insomnia and nightmares. Such side effects have limited the practical usefulness of orally administered clonidine. Despite the usefulness of clonidine in the treatment of hypertension, the regimen of administration required by the pharmacokinetic profile of the drug resulted in quite wide fluctuations in plasma concentrations, even at steady state. (Fujimura A., et al., J. Clin. Pharmacol. 1994; 34:260-265). It has been shown that many of the adverse events (AEs) observed during oral clonidine administration were related to its high peak plasma concentrations. (Lowenthal D T., J. Cardiovasc. Pharmacol., 1980; 2(suppl.):S29-S37).

The pharmacokinetic profile and relationship between plasma levels and AEs necessitate frequent dosing and result in a “roller coaster” effect characterized by “peak” AE of sedation and trough AE of rebound hypertension. In an effort to address the “roller coaster” issue, a 7-day patch formulation for clonidine (marketed under the brand name Catapres-TTS) was developed. Early studies showed that transdermal administration of clonidine was safe and effective in controlling hypertension (Weber, M A, et al., Arch. Intern. Med., 1984; 144(6):1211-1213. In addition, these studies suggested a milder AE profile for the patch formulation than for oral clonidine with reduced sedation and lack of rebound hypertension. The patch, however, had severe limitations. First, localized skin reactions such as erythema, pruritus and localized vesiculation was observed in over 50% of patients. In a large database of exposure to transdermal clonidine reviewed by the FDA, these skin reactions led to discontinuation of treatment in 19% of patients. Furthermore, the label cautions that in patients who develop an allergic reaction to transdermal clonidine, substitution of oral clonidine may also elicit an allergic reaction including generalized rash, urticaria or angioedema. Another problem that has plagued the patch is poor adhesiveness necessitating the use of an adhesive overlay.

A capsule containing microcapsule having a range of differing release profiles has been used as a sustained release formulation of clonidine. (Mancia, G. et al., J. Cardiovasc. Pharmacol., 1981; 3:1193-1202; Fyhrquist, F., Intl. J. Clin. Pharmacol., Therapy and Toxicol., 1983; 21:12:634-636). This formulation is known as Catapresan-Perlonget and is available in Europe. Typically, the sustained release formulation contains different membrane coated nuclei of the drug. One nuclei releases the drug rapidly while the others release more slowly over 3 or 6 hours, respectively. (Mancia).

For the foregoing reasons, there is a need for drug formulations, such as low dosage drug formulations, that are capable of stable therapeutic dosage profiles by providing an extended serum level concentration of active for an extended period in order to avoid possible “peak and trough” side effects (effectiveness at peak serum levels and rebound exacerbation of symptoms at trough levels).

### BRIEF DESCRIPTION OF THE DRAWINGS

FIGS. 1A and 1B depict the mean Clonidine concentration-time profiles after administration of CLONICEL®-Fasted (Treatment A), CLONICEL®-Fed (Treatment B) and Catapres-Fasted (Treatment C).

FIG. 2 is a graphical representation of predicted area under the curve (AUC) blood plasma levels of an exemplary extended release composition embodiment disclosed herein in comparison with an immediate release composition.

FIG. 3 depicts the mean clonidine concentration-time profiles by treatment group for days 23 and 25. Average concentrations for the 3 treatment groups ranged from approximately 400 pg/mL to 1800 pg/mL. Plasma concentrations increased proportionately with increase in dose, stayed fairly even throughout the inter-dosing interval, and were very similar between Days 23 and 25.

FIG. 4 depicts the mean (±SD) steady-state trough clonidine concentrations on days 23, 25 and 26. The relationship between dose and derived PK parameters was explored by plotting Cₘₐₓ, Cₘᵢₙ, AUCτ, and CL/F values for Day 25 as a function of the administered dose. As the figure shows, the three exposure parameters appeared to increase proportionately with the dose, and CL/F decreased slightly over the dosing range.

FIG. 5 depicts a sigmoidal Eₘₐₓ relationship between effect on systolic blood pressure and clonidine Cₘₐₓ.

FIGS. 6A, 6B and 6C depict the mean daytime SBP (systolic blood pressure), DBP (diastolic blood pressure), and change from Baseline to day 26.

FIGS. 7A and 7B depict the mean daytime systolic and diastolic blood pressure observations at Baseline and for Days 26 to 28. As is evident from the data, both SBP and DBP daytime values gradually returned to Baseline levels over the 48 hours post-dosing without overshoot even though study medication had been withdrawn abruptly.

FIGS. 8A, 8b and 8C depict the mean SBP profiles by treatment at baseline and day 26.

FIGS. 9A, 9B and 9C depict the mean DBP profiles by treatment at baseline and day 26.

FIGS. 10A, 10B and 10C depict the mean heart rate profiles by treatment at baseline and day 26.

FIG. 11 depicts the escalating titration schedule used to reach steady-state plasma concentrations of clonidine.

FIG. 12 depicts mean clonidine concentration-time profiles after administration of CloniDAY 1×0.4 mg-Fasted (Treatment A), CloniDAY 1×0.4 mg-Fed (Treatment B), and CloniDAY 4×0.1 mg-Fasted (Treatment C).

FIG. 13 depicts mean clonidine concentration-time profiles after administration of CloniDAY 0.2 mg-QD (Treatment A) on Days 6 and 7 excluding subjects 108 and 110.

FIG. 14 depicts mean steady state clonidine concentration-time profiles after administration of CloniDAY 0.2 mg-QD (Treatment A) and Sympres 0.1 mg-BID (Treatment B) excluding subjects 108 and 110.

FIG. 15 depicts mean and individual predose clonidine concentrations after administration of CloniDAY 0.2 mg-QD (Treatment A) excluding subjects 108 and 110. Mean predose concentrations are shown as a dotted line.

FIG. 16 depicts mean and individual predose clonidine concentrations after administration of Sympres 0.1 mg-BID (Treatment B) excluding subjects 108 and 110. Mean predose concentrations are shown as a dotted line

### SUMMARY OF THE INVENTION

Described herein is an oral dosage form comprising: (a) an α₂-adrenergic receptor agonist in an amount between 0.001 wt % and 0.5 wt % of said oral dosage form; and (b) a pharmaceutically acceptable hydrophilic matrix comprising: (i) at least one hydroxypropyl methylcellulose ether in an amount between 20 wt % and 80 wt % of the oral dosage form; (ii) at least one of starch, lactose, or dextrose in an amount between 20 wt % and 80 wt % of the oral dosage form; and (iii) a metal alkyl sulfate; wherein after administration of the oral dosage form no more than once about every 12 hours (e.g., no more than once about every 24 hours) to a subject having a steady state plasma concentration of the co-adrenergic receptor agonist, the agonist's plasma concentration peak-to-trough ratio is no greater than about 1.9.

In another embodiment, a method of treating adrenergic dysregulation in a subject in need thereof is disclosed. The method comprises orally administering to the subject no more than once about every 12 hours (e.g., no more than once about every 24 hours) the oral dosage formulation described herein, which provides a plasma peak-to-trough ratio no greater than about 1.9, wherein the adrenergic dysregulation is treated.

In a further embodiment, the invention relates to an oral dosage form comprising an α₂-adrenergic receptor agonist; wherein after administration of said dosage form no more than once about every 24 hours to a subject having a steady state plasma concentration of said α₂-adrenergic receptor agonist, the AUC per 0.2 mg dose is about 9,000 to about 17,000 h·pg/mL.

In an additional embodiment, the invention relates to an oral dosage form comprising an α₂-adrenergic receptor agonist; wherein after administration of said dosage form no more than once about every 12 hours to a subject having a steady state plasma concentration of said α₂-adrenergic receptor agonist, the AUC per 0.1 mg dose is about 5,500 to about 9,500 h·pg/mL.

### DETAILED DESCRIPTION OF THE INVENTION

The present invention will now be described in more detail, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of skill in the art to which this invention belongs. The terminology used in the description of the invention herein is for the purpose of describing particular embodiments only and is not intended to be limiting of the invention. All publications, patent applications, patents, patent publications and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed.

As used in the description of the invention and the appended claims, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of compound, dose, time, temperature, enzymatic activity or other biological activity and the like, is meant to encompass variations of +20%, ±10%, ±5%, ±1%, ±0.5%, or even ±0.1% of the specified amount.

As used herein, the transitional phrase “consisting essentially of” is to be interpreted as encompassing the recited materials or steps “and those that do not materially affect the basic and novel characteristic(s)” of the claimed invention (e.g., pharmacokinetic characteristics). See, In re Herz, 537 F.2d 549, 551-52, 190 U.S.P.Q. 461, 463 (CCPA 1976) (emphasis in the original); see also MPEP §2111.03. Thus, the term “consisting essentially of” as used herein should not be interpreted as equivalent to “comprising.”

In one embodiment, an oral dosage form is provided. The dosage form comprises, consists essentially of, or consists of an α₂-adrenergic receptor agonist in an amount between 0.001 wt % and 0.5 wt % of the oral dosage form; a pharmaceutically acceptable hydrophilic matrix comprising a mixture of at least one hydroxypropyl methylcellulose ether in an amount between 20 wt % and 80 wt % of the oral dosage form; at least one of starch, lactose, or dextrose in an amount between 80 wt % and 20 wt % of the oral dosage form; a release-retardant of a metal alkyl sulfate; and optionally a metal stearate and/or colloidal silica.

In another embodiment, an oral dosage form is disclosed, wherein the oral dosage form comprises, consists essentially of, or consists of: (a) an α₂-adrenergic receptor agonist in an amount between 0.001 wt % and 0.5 wt % of said oral dosage form; and (b) a pharmaceutically acceptable hydrophilic matrix comprising: (i) at least one hydroxypropyl methylcellulose ether in an amount between 20 wt % and 80 wt % of the oral dosage form; (ii) at least one of starch, lactose, or dextrose in an amount between 20 wt % and 80 wt % of the oral dosage form; and (iii) a metal alkyl sulfate; wherein after administration of the oral dosage form no more than once about every 12 hours (e.g., no more than once about every 24 hours) to a subject having a steady state plasma concentration of the α₂-adrenergic receptor agonist, the agonist's plasma concentration peak-to-trough ratio is no greater than about 1.9.

In another embodiment, disclosed is an oral dosage form comprising, consisting essentially of, or consisting of: (a) an α₂-adrenergic receptor agonist in an amount between 0.001 wt % and 0.5 wt % of the oral dosage form; and (b) a pharmaceutically acceptable hydrophilic matrix comprising: (i) at least one hydroxypropyl methylcellulose ether in an amount between 20 wt % and 80 wt % of said oral dosage form; (ii) at least one of starch, lactose, or dextrose in an amount between 20 wt % and 80 wt % of the oral dosage form; and (iii) a metal alkyl sulfate; wherein after a first administration to a subject of the dosage form, the agonist's plasma concentration peak-to-trough ratio is no greater than about 1.9 for any subsequent administration of the dosage form, wherein the subsequent administration is no more than once about every 12 hours (e.g., no more than once about every 24 hours).

In yet another embodiment, a solid oral dosage form for treating and/or reducing an adrenergic dysregulation condition in a subject in need thereof is disclosed. The solid oral dosage form comprises, consists essentially of, or consists of, a) an α₂-adrenergic receptor agonist; b) a pharmaceutically acceptable hydrophilic matrix providing a release rate of the α₂-adrenergic receptor agonist; and c) a release-retardant in an amount such that the release rate of the α₂-adrenergic receptor agonist from the hydrophilic matrix is decreased.

In another embodiment, a method of treating an adrenergic dysregulation condition in a subject in need thereof is disclosed. The method comprises, orally administering to a subject a formulation comprising an effective amount of an co-adrenergic receptor agonist, the α₂-adrenergic receptor agonist admixed within a pharmaceutically acceptable hydrophilic matrix comprising a release-retardant; and providing an extended release rate of the α₂-adrenergic receptor agonist from the formulation; wherein the extended release rate of the α₂-adrenergic receptor agonist from the pharmaceutically acceptable hydrophilic matrix with the release-retardant admixed therein is less than a release rate for the α₂-adrenergic receptor agonist from the pharmaceutically acceptable hydrophilic matrix without the release-retardant admixed therein. In this embodiment, the method can further include, providing (i) a plasma concentration level of the α₂-adrenergic receptor agonist from the pharmaceutically acceptable hydrophilic matrix; and (ii) a peak plasma level concentration of the α₂-adrenergic receptor agonist from the pharmaceutically acceptable hydrophilic matrix; wherein the plasma concentration level of co-adrenergic receptor agonist from the pharmaceutically acceptable hydrophilic matrix with the release-retardant admixed therein provides an extended plasma concentration level of the α₂-adrenergic receptor agonist and a reduced peak plasma level concentration of the α₂-adrenergic receptor agonist than a pharmaceutically acceptable hydrophilic matrix and the α₂-adrenergic receptor agonist without the release-retardant admixed therein. In this embodiment, the extended release rate of the α₂-adrenergic receptor agonist from the pharmaceutically acceptable hydrophilic matrix is zero-order to first-order.

In another embodiment, a method of treating adrenergic dysregulation in a subject in need thereof is provided. The use of the present formulations may provide for improved therapies for symptoms manifesting from adrenergic dysregulation conditions by systemic control of catecholamines. The method comprises orally administering to the subject a formulation comprising an effective amount of an α₂-adrenergic receptor agonist the α₂-adrenergic receptor agonist admixed within a pharmaceutically acceptable hydrophilic matrix comprising a release-retardant. The method provides an extended release rate of the α₂-adrenergic receptor agonist. The extended release rate of the α₂-adrenergic receptor agonist from the pharmaceutically acceptable hydrophilic matrix with the release-retardant admixed therein is less than a release rate for the α₂-adrenergic receptor agonist from the pharmaceutically acceptable hydrophilic matrix without the release-retardant admixed therein.

In yet another embodiment, disclosed is a method of treating adrenergic dysregulation in a subject in need thereof, comprising: administering an oral dosage form as described herein to a subject no more than once about every 12 hours (e.g., no more than once about every 24 hours), wherein the subject has a steady state plasma concentration of the α₂-adrenergic receptor agonist, and wherein after the administering, the agonist's plasma concentration peak-to-trough ratio is no greater than about 1.9; wherein the adrenergic dysregulation is treated.

The α₂-adrenergic receptor agonist can be any compound or composition of matter that binds to the α₂-adrenergic receptor of a cell to produce a central α-adrenergic stimulation within the cell. Examples of α₂-adrenergic receptor agonists include epinephrine, noradrenaline, isoprenaline, clonidine, guanfacine, lofexidine, xylazine, or their salts. In preferred embodiments, the agonist is clonidine or a pharmaceutically acceptable salt thereof. Most preferably, the agonist is clonidine hydrochloride. The aforementioned agonists may be supplied as pure compounds, or in a form of a pharmaceutically active salt, isomer, a racemic mixture, or in any other chemical form or combination that, under physiological conditions, provides for therapeutically effective treatment of adrenergic dysregulation.

As used herein, “clonidine” refers to a 9-carbon, two-ringed imidazoline derivative. The term “clonidine” denotes generally one or more of 2,6-dichloro-N-2-imidazolidinylidene benzeneamine, or benzeneamines structurally and functionally related thereto that are described in U.S. Pat. No. 3,454,701. U.S. Pat. No. 3,454,701, is incorporated herein by reference for its disclosure of such structurally and functionally related benzeneamines. As used herein, lofexidine refers to 2-[1-(2,6-dichlorophenoxy)ethyl]-4,5-dihydro-1H-imidazole or structurally and functionally related imidazoles. As used herein, xylazine refers to 2-(2,6-dimethylphenylamino)-5,6-dihydro-4H-thiazine or structurally and functionally related thiazines. With respect to the preferred embodiments of the present invention, the term “clonidine” denotes 2,6-dichloro-N-2-imidazolidinylidene benzeneamine, and its various tautomers and rotomers. In a preferred embodiment, it has the following structure:

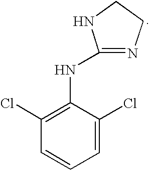

The amount of α₂-adrenergic receptor agonists that is included per oral dosage form may vary widely. For example, the therapeutically effective dose range for the α₂-adrenergic receptor agonist clonidine is about 0.025 mg to about 0.8 mg per dosage form for most of the symptoms of the clinical disorders listed above. The therapeutically effective dose range of about 0.025 mg to about 0.8 mg per dosage form typically controls most of the symptoms of adrenergic dysregulation. For oral dosage forms to be administered no more than once every 12 hours, the amount of clonidine or a pharmaceutically acceptable salt thereof can be in the range of about 0.025 to about 0.4 mg per dosage form, e.g., about 0.05 to about 0.2 mg per dosage form, e.g., about 0.1 mg per dosage form. For oral dosage forms to be administered no more than once every 24 hours, the amount of clonidine or a pharmaceutically acceptable salt thereof can be in the range of about 0.1 to about 0.8 mg per dosage form, e.g., about 0.2 to about 0.6 mg per dosage form, e.g., about 0.3 to about 0.5 mg per dosage form, e.g., about 0.4 mg per dosage form.

Adrenergic dysregulation refers generally to conditions of cardiovascular, analgesic, neurologic/psychiatric, or gastrointestinal/renal origin resulting from abnormal neuronal activation or secretion of adrenaline and/or noradrenaline. By way of example, cardiovascular conditions include those conditions manifested in hypertension, atrial fibrillation, congestive heart failure, and orthostatic hypotension. Analgesic conditions include those conditions manifested in intraoperative and postoperative pain, intractable cancer pain, headaches, labor pain, and reflex sympathetic dystrophy. Neurologic/psychiatric conditions include those conditions manifested in akathisia, peripheral neuropathy, neuropathic orofacial pain, diabetic gastroparesis, essential tremor, postepidural shivering, postanesthesia shivering, restless legs syndrome, hypertonicity, hyperkinetic movement disorders, Tourette's syndrome, substance withdrawal, acute anorexia nervosa, attention-deficit/hyperactivity disorder (ADHD), conduct disorder, bipolar disorder, aggression, narcolepsy, panic disorder, posttraumatic stress disorder, sleep disorders, social phobia, and schizophrenia. Gastrointestinal/renal conditions include those conditions manifested in ulcerative colitis and proctitis, emesis, and cyclosporine-induced nephrotoxicity. Endocrine/hormonal conditions include those conditions manifested in hyperthyroidism, growth delay in children, excessive sweating, post-menopausal flushing, and hot flashes. In certain embodiments, the conditions are ADHD, hypertension, or post-menopausal flushing and hot flashes.

Attention Deficit Hyperactivity Disorder and ADHD refer to any etiological or pathological symptom associated with the disorder. Such symptoms and etiology include inattention, hyperactivity and impulsivity. Generally, a subject will exhibit significant impairment occurring in at least two settings and/or consistently display such characteristic behaviors over an extended period of time. The teens also include Attention Deficit Disorder (ADD).

Hypertension refers generally to any etiological or pathological symptom manifested in blood pressure that is chronically elevated. Such symptoms include low-renin levels, insulin resistance, sleep apnea, excess serum sodium levels, obesity and genetic disposition.

Useful amounts of agonist present in the formulation are between about 0.001 wt % and 0.5 wt % of the dosage form. For oral dosage forms to be administered no more than once every 12 hours, the amount can be between about 0.01 wt % and about 0.3 wt %, e.g., between about 0.05 wt % and 0.2 wt %, e.g., between about 0.05 wt % and about 0.1 wt %, e.g., about 0.08 wt %. For oral dosage forms to be administered no more than once every 24 hours, the amount can be between about 0.1 wt % and about 0.5 wt %, e.g., between about 0.2 wt % and about 0.5 wt %, e.g., between about 0.2 wt % and about 0.4 wt %, e.g., about 0.3 wt %.

Useful amounts of the hydroxypropyl methylcellulose ether(s) (as a total of all of the hydroxypropyl methylcellulose ethers present in the formulation) are between about 20 wt % and 80 wt % of the dosage form. Preferably, the amount is between about 30 wt % and 50 wt %. More preferably, the amount is between about 40 wt % and 60 wt %. Most preferably, the amount is between about 20 wt % and 40 wt %, or 60 wt % and 80 wt %. In some embodiments, the amount is between about 35 wt % and about 42 wt %, e.g., about 38 wt %.

The phrase “at least one of starch, lactose, or dextrose” encompasses any combination of the three components, e.g., starch and lactose, starch and dextrose, lactose and dextrose, or all three. Useful amounts of starch, lactose or dextrose (as a total of starch, lactose or dextrose together) are between about 20 wt % and 80 wt % of the dosage form. Preferably, the amount is between about 50 wt % and 70 wt %. More preferably, the amount is between about 40 wt % and 60 wt %. Most preferably, the amount is between about 20 wt % and 40 wt %, or 60 wt % and 80 wt %. In some embodiments, the amount is between about 55 wt % and about 60 wt %. In embodiments in which both starch and lactose are present, the amount of starch can be between about 20 wt % and about 40 wt %, e.g., between about 25 wt % and about 35 wt %, e.g., between about 28 wt % and about 30 wt %, and the amount of lactose can be between about 20 wt % and about 40 wt %, e.g., between about 25 wt % and about 35 wt %, e.g., between about 27 wt % and about 32 wt %.

Useful amounts of metal alkyl sulfate are between about 1 wt % and 8 wt % of the dosage for n. Preferably, the amount is between about 1 wt % and 7 wt %. More preferably, the amount is between about 2 wt % and 6 wt %. For oral dosage forms to be administered no more than once every 12 hours, the amount can be between about 1 wt % and about 3 wt %, e.g., between about 1.5 wt % and about 2.5 wt %, e.g., about 2 wt %. For oral dosage forms to be administered no more than once every 24 hours, the amount can be between about 3 wt % and about 7 wt %, e.g., between about 4 wt % and about 6 wt %, e.g., about 5 wt %. Metal alkyl sulfates are known in the art and include, for example, ammonium lauryl sulfate, magnesium laureth sulfate, sodium dodecyl sulfate (sodium lauryl sulfate), sodium laureth sulfate, sodium myreth sulfate and sodium pareth sulfate. Preferably, the metal alkyl sulfate is sodium lauryl sulfate (SLS).

Useful amounts of a metal stearate and/or colloidal silica (as a total of metal stearate and colloidal silica together) are between about 0.1 wt % and about 5 wt %, e.g., between about 0.1 wt % and about 2 wt %, e.g., between about 0.5 wt % and about 1.5 wt %, e.g., about 1 wt %. When both a metal stearate and a colloidal silica are present, the amount of metal stearate can be between about 0.1 wt % and about 3 wt %, e.g., between about 0.5 wt % and about 1 wt %, e.g., about 0.5 wt %, and the amount of colloidal silica can be between about 0.01 wt % and about 2 wt %, e.g., between about 0.1 wt % and about 0.5 wt %, e.g., about 0.2 wt %.

In one embodiment, the pharmaceutically acceptable hydrophilic matrix comprises, consists essentially of, or consists of: (i) at least one hydroxypropyl methylcellulose ether in an amount between 20 wt % and 50 wt % of said oral dosage form; (ii) at least one of starch, lactose, or dextrose in an amount between 40 wt % and 70 wt % of said oral dosage form; and (iii) a metal alkyl sulfate in an amount between about 1 wt % and about 7 wt %.

In one embodiment, the pharmaceutically acceptable hydrophilic matrix comprises, consists essentially of or consists of: (i) at least one hydroxypropyl methylcellulose ether in an amount between 30 wt % and 45 wt % of said oral dosage form; (ii) at least one of starch, lactose, or dextrose in an amount between 50 wt % and 60 wt % of said oral dosage form; and (iii) a metal alkyl sulfate in an amount between about 5 wt % and about 7 wt %.

In one embodiment, the oral dosage form comprises, consists essentially of, or consists of (a) an α₂-adrenergic receptor agonist in an amount between 0.001 wt % and 0.5 wt % of said oral dosage form; and (b) a pharmaceutically acceptable hydrophilic matrix comprising: (i) at least one hydroxypropyl methylcellulose ether in an amount between 30 wt % and 45 wt % of said oral dosage form; (ii) at least one of starch, lactose, or dextrose in an amount between 50 wt % and 60 wt % of said oral dosage form; and (iii) a metal alkyl sulfate in an amount between about 4 wt % and about 6 wt %; and (c) a metal stearate and colloidal silica in an amount between about 0.1 wt % and about 2 wt %.

In one embodiment for 12 hour dosing, the oral dosage form comprises, consists essentially of, or consists of (a) clonidine hydrochloride in an amount of about 0.08 wt % of said oral dosage form; and (b) a pharmaceutically acceptable hydrophilic matrix comprising: (i) at least one hydroxypropyl methylcellulose ether in an amount of about 38 wt % of said oral dosage form; (ii) starch in an amount of about 29 wt % of said oral dosage form and lactose in an amount of about 30 wt % of said oral dosage form; and (iii) a metal alkyl sulfate in an amount of about 2 wt %; and (c) a metal stearate in an amount of about 1 wt % and colloidal silica in an amount of about 0.2 wt %.

In one embodiment for 24 hour dosing, the oral dosage form comprises, consists essentially of, or consists of (a) clonidine hydrochloride in an amount of about 0.3 wt % of said oral dosage form; and (b) a pharmaceutically acceptable hydrophilic matrix comprising: (i) at least one hydroxypropyl methylcellulose ether in an amount of about 38 wt % of said oral dosage form; (ii) starch in an amount of about 28 wt % of said oral dosage form and lactose in an amount of about 27 wt % of said oral dosage form; and (iii) a metal alkyl sulfate in an amount of about 5 wt %; and (c) a metal stearate in an amount of about 1 wt % and colloidal silica in an amount of about 0.2 wt %.

In particular embodiments, the α₂-adrenergic receptor agonist is clonidine hydrochloride, the hydroxypropyl methylcellulose ether is hypromellose type 2208 (e.g., Methocel K100 premium), the starch is partially pregelatinized starch, the lactose is lactose monohydrate, the metal alkyl sulfate is sodium lauryl sulfate, and the metal stearate is magnesium stearate, or any combination thereof.

The useful and preferred values of the dosage form are also useful and preferred values when used in the methods described herein.

The peak-to-trough ratio is defined as the highest blood plasma concentration divided by the lowest blood plasma concentration within a dosing interval. The dosing interval is the time from the administration of a dose to the time of the next administration. Determining the time at which blood plasma can be measured to ensure the highest and lowest concentrations are determined is within the purview of a skilled artisan.

Minimizing the fluctuation in plasma concentration yields beneficial results. The leveling of the blood plasma concentrations over a dosing interval and, consequently, over the course of potentially long-term therapy provides the consistent plasma levels necessary to treat or ameliorate α₂-adrenergic dysregulation. A useful peak-to-trough ratio is no greater than about 1.9. Preferably, the ratio is no greater than about 1.6. Also preferred is a ratio between about 1.1 and about 1.6. Most preferably, the ratio is between about 1.3 and 1.6. The most preferred ratio is about 1.4. In certain embodiments, the ratio is no greater than about 1.1, 1.2, 1.3, 1.4, 1.5, 1.6, 1.7, 1.8, 1.9, or 2.0. The lower the ratio, the less fluctuation and, therefore, there are fewer associated side effects.

Steady-state is defined as the plasma concentration levels after about five half-lives. Thus, steady-state is reached at different times for different actives. Clonidine's half-life is about 12 to 17 hours. Therefore, clonidine steady-state is reached at about day four.

The hydrophilic matrix provides for a controlled pharmacokinetic release profile of the α₂-adrenergic receptor agonist. The hydrophilic matrix provides for a zero- to first-order release profile of the α₂-adrenergic receptor agonist. When using a combination of components for the hydrophilic matrix, the ratio of the components may influence the release profile of the α₂-adrenergic receptor agonist from the matrix. For a low dose α₂-adrenergic receptor agonist (for example, clonidine) the ratio of the components may not be predictable or determinable. By adjusting the amount of hydrophilic polymer and/or release-retardant compared to the α₂-adrenergic receptor agonist, the release profile of the α₂-adrenergic receptor agonist may be adjusted or more easily tailored to a particularly advantageous therapeutically effective profile. By releasing the drug over a longer period of time, therapeutically effective profiles of up to and including 24 hour dosing of the α₂-adrenergic receptor agonist is provided with reduction or elimination of undesirable side effects, such as hyperarousal. More specifically, the formulation disclosed herein provides minimal fluctuation of plasma concentrations of an α₂-adrenergic receptor agonist, such as clonidine at steady-state.

The data provided herein show that the present formulation provides plasma concentrations at steady state that are predictable from day to day. Further, when measured on two days separated by 48 hours, the concentrations were very similar on a patient by patient basis indicating consistent performance between individual drug units. The narrow peak-to-trough plasma concentrations provide a therapeutically effective amount of active without the roller-coaster effect that comes with the high peak-to-trough fluctuations seen in prior art formulations. The present formulation provides blood levels achieved from the clonidine patch in an oral sustained-release tablet. In its review of data from the clonidine patch, FDA noted that the peak to trough ratio in steady state concentrations observed with the clonidine patch averaged about 1.33 whereas the corresponding fluctuation with the immediate release clonidine tablet averaged 2.10. Data from the present study show average ratios with CLONICEL® of about 1.4 to about 1.5 when administered every 12 hours, which are ratios that are much closer to the clonidine patch than to the immediate release tablet. When administered every 24 hours, the average ratio with CloniDAY is about 1.9, substantially less that the immediate release tablet. The data further shows that a 24 hour dosage form exhibits similar pharmacokinetic characteristics to a 12 hour dosage form, including overall exposure levels and peak concentrations.

In certain aspects, the formulations of the invention provide advantageous pharmacokinetic characteristics for the delivery of α₂-adrenergic receptor agonists. In one embodiment, the invention provides an oral dosage form comprising an α₂-adrenergic receptor agonist; wherein after administration of said dosage for in no more than once about every 12 hours to a subject having a steady state plasma concentration of said α₂-adrenergic receptor agonist, the AUC per 0.1 mg dose is about 5,500 to about 9,500 h·pg/mL, e.g., about 6,500 to about 8,500 h·pg/mL, e.g., about 7,000 to about 8,000 h·pg/mL or any range therein.

In another embodiment, the invention provides an oral dosage form comprising an α₂-adrenergic receptor agonist; wherein after administration of said dosage form no more than once about every 12 hours to a subject having a steady state plasma concentration of said α₂-adrenergic receptor agonist, the Cₘₐₓ is about 500 to about 900 pg/mL after administration of a 0.1 mg dose of the agonist, e.g., about 600 to about 800 pg/mL, e.g., about 700 to about 750 pg/mL or any range therein.

In another embodiment, the invention provides an oral dosage form comprising an α₂-adrenergic receptor agonist; wherein after administration of said dosage form no more than once about every 12 hours to a subject having a steady state plasma concentration of said co-adrenergic receptor agonist, the Tₘₐₓ of the agonist after a single dose is about 4 to about 6 hours, e.g., about 4.5 to about 5.5 hours or any range therein.

In one embodiment, the invention provides an oral dosage form comprising an α₂-adrenergic receptor agonist; wherein after administration of the dosage form no more than once about every 24 hours to a subject having a steady state plasma concentration of the α₂-adrenergic receptor agonist, the AUC per 0.2 mg dose is about 9,000 to about 17,000 h·pg/mL, e.g., about 10,000 to about 15,000 h·pg/mL, e.g., about 11,000 to about 14,000 h·pg/mL, or any range therein.

In another embodiment, the invention provides an oral dosage form comprising an α₂-adrenergic receptor agonist; wherein after administration of the dosage form no more than once about every 24 hours to a subject having a steady state plasma concentration of the co-adrenergic receptor agonist, the Cₘₐₓ is about 500 to about 900 pg/mL after administration of a 0.2 mg dose of the agonist, e.g., about 600 to about 800 pg/mL, e.g., about 650 to about 750 pg/mL, or any range therein.

In another embodiment, the invention provides an oral dosage form comprising an α₂-adrenergic receptor agonist; wherein after administration of the dosage form no more than once about every 24 hours to a subject having a steady state plasma concentration of the α₂-adrenergic receptor agonist, the Tₘₐₓ of the agonist after a single dose is about 4 to about 10 hours, e.g., about 5 to about 8 hours, e.g., about 6 to about 7 hours, or any range therein.

The compositions of the invention can exhibit any one or more of the pharmacokinetic parameters described above, e.g., one or more of AUC, Cₘₐₓ, and/or Tₘₐₓ.

The term “hydrophilic matrix” refers to one or more natural or synthetic materials that are hydrophilic, but not necessarily water-soluble. Examples of a hydrophilic matrix include polymer or polymers having affinity for absorbing water such as cellulose ethers (e.g., hydroxypropyl methylcellulose), mono or disaccharides (for example, dextrose or lactose), starch, derivatives thereof, alone or in combination.

The term “starch” refers generally to a mixture of polysaccharides of plant origin, the polysaccharides including amylose and amylopectin. Starch includes, for example, sorghum, plantain and corn starches. The term “starch” includes material that has been chemically- and/or mechanically-processed in the presence of water and subsequently dried. By way of example, the term “starch” includes pregelatinized starch, which encompasses completely chemically- and/or mechanically-processed starch or mixtures of partially and completely chemically- and/or mechanically-processed starches. Partially pregelatinized starch includes, for example a mixture comprising one or more of a modified starch and one or more of an unmodified starch, each starch independently selected from sorghum, plantain and corn starches.

The term “lactose” refers to a chemical compound comprising a β-D-galactose and a β-D-glucose molecule linked through a β₁₋₄ glycosidic chemical bond, and derivatives thereof. Lactose may be provided in any form, e.g., spray dried, modified spray dried, or hydrated (e.g., lactose monohydrate).

The term “dextrose” refers to a chemical compound comprising a glucose molecule and derivatives thereof. D-glucose is preferred. Dextrose may be provided in any form, e.g., spray dried, modified spray dried, or hydrated.

As used herein, the term “treatment” and its grammatical equivalents refer to the alleviation or elimination of etiological or pathological symptoms and include, for example, the elimination of such symptom causation either on a temporary or permanent basis, or to alter or slow the appearance of such symptoms or symptom worsening. For example, the term “treatment” includes alleviation or elimination of causation of symptoms associated with, but not limited to, adrenergic dysregulation or its related-complications described herein. Treatment includes the prevention of the associated condition.

“Therapeutically effective” refers qualitatively to the amount of an agent or agents in combination for use in adrenergic dysregulation therapy that is nontoxic but sufficient to provide the desired effect that will achieve the goal of preventing, or improvement in the severity of the symptoms. Adrenergic dysregulation or its related complication symptoms is considered prevented or improved if any benefit is achieved, irrespective of the absolute magnitude of the amelioration or improvement. For example, any reduction in blood pressure of a subject suffering from hypertension would be considered an ameliorated symptom. Likewise, any inhibition or suppression of inattention, hyperactivity and impulsivity would also be considered amelioration of ADHD. Furthermore, any reduction or elimination in side-effects such as “peak and trough” side effects of transient sedation at peak serum levels and rebound exacerbation of symptoms at trough levels of a subject on an ADHD therapy is considered an ameliorated symptom.

As used herein, “therapeutically effective amount” refers to an amount of an active agent. The therapeutically effective amount varies according to the patient's sex, age and weight, the route of administration, the nature of the condition and any treatments, which may be associated therewith, or any concurrent related or unrelated treatments or conditions of the patient. In determining the effective amount or dose, a number of factors are considered by the attending diagnostician, including, but not limited to, the potency and duration of action of the compounds used, the nature and severity of the illness to be treated, as well as the sex, age, weight, general health and individual responsiveness of the patient to be treated, and other relevant circumstances. Therapeutically effective amounts may be determined without undue experimentation by any person skilled in the art or by following the exemplary guidelines set forth in this application.

As used herein, the term “subject” for purposes of treatment or prevention includes any subject, and preferably is a subject who is in need of an adrenergic dysregulation treatment, or who needs treatment of an adrenergic dysregulation related complication. For purposes of prevention, the subject is any subject, and preferably is a subject that is at risk for, or is predisposed to, an adrenergic dysregulation condition or its related complications. The subject is typically an animal, more typically is a mammal. Preferably, the mammal is a human, horse, dog or cat.

As used herein, the terms “subject in need thereof” and grammatical equivalents refer to any subject who is suffering from or is predisposed to an adrenergic dysregulation condition or its related complications. The terms include any subject that requires a lower dose of therapeutic agents. In addition, the terms include any subject who requires a reduction in the side-effects of a therapeutic agent. Furthermore, the terms include any subject who requires improved tolerability to any therapeutic agent for an adrenergic dysregulation therapy.

The pharmaceutically acceptable hydrophilic matrix as herein disclosed may comprise polysaccharides, for example, cellulose derivatives. Examples of such polysaccharides include alkylcelluloses, such as, methylcellulose; hydroxyalkylcelluloses, for example, hydroxymethylcellulose, hydroxyethylcellulose, hydroxypropylcellulose and hydroxybutylcellulose; hydroxyalkyl alkylcelluloses, such as, hydroxyethyl methylcellulose and hydroxypropyl methylcellulose; carboxyalkylcelluloses, such as, carboxymethylcellulose; alkali metal salts of carboxyalkylcelluloses, such as, sodium carboxymethylcellulose; carboxyalkylalkylcelluloses, such as, carboxymethylethylcellulose; carboxyalkylcellulose esters; other natural, semi-synthetic, or synthetic polysaccharides, such as, alginic acid, alkali metal and ammonium salts thereof. By way of example, the pharmaceutically acceptable hydrophilic matrix is a cellulose ether derivative. The cellulose ether derivative is a hydroxypropyl methylcellulose.

The hydrophilic matrix may include hydroxypropyl methyl cellulose (HPMC). Different viscosity grades of HPMC are commercially available. The HPMC may have a hydroxypropoxyl substitution of from about 7 to about 12 weight percent, a methoxyl substitution of from about 28 to about 30 weight percent, a number average molecular weight of about 86,000 and a 2% aqueous solution viscosity of about 4000 cps. The HPMC may have a hydroxypropoxyl substitution of from about 7 to about 12 weight percent, a methoxyl substitution of from about 19 to about 24 weight percent, a number average molecular weight of about 246,000 and a 2% aqueous solution viscosity of about 100,000 cps. Mixtures of the above HPMC's may be used. The hydrophilic matrix may comprise a hydroxypropyl methylcellulose such as Methocel®, which is manufactured by the Dow Chemical Company, U.S.A. In one embodiment, the HPMC is hypromellose type 2208 (e.g., Methocel® K100 premium).

The hydrophilic matrix may also comprise polyacrylic acids and the salts thereof, crosslinked acrylic acid-based polymers, for example CARBOPOL™ polymers (Lubrizol Corp., Wickliffe, Ohio); polymethacrylic acids and the salts thereof, methacrylate copolymers; polyvinylalcohol; polyvinylpyrrolidone, copolymers of polyvinylpyrrolidone with vinyl acetate; combinations of polyvinylalcohol and polyvinylpyrrolidone; polyalkylene oxides such as polyethylene oxide and polypropylene oxide and copolymers of ethylene oxide and propylene oxide.

The HPMC may be admixed with additional hydrophilic polymers, for example, starch, pregelatinized starch, monosaccharides, or disaccharides. By way of example, the HPMC may be admixed with dextrose, sucrose, lactose, lactulose, trehalose, maltose, mannitol, sorbitol or combinations thereof. For example, the lactose or lactose monohydrate may be used. Different grades of lactose may be used. The lactose is a modified spray-dried lactose monohydrate (316 Fast Flow, WI). Other lactose monohydrates, may also be used. The particles of lactose monohydrate may be such that 98% (w/w) of the particles are smaller than 850 μm. The hydrophilic matrix may comprise a HPMC admixed with a partially gelatinized starch or a combination/admixture of lactose and partially gelatinized starch. By way of example, Starch 1500® NF (Colorcon, West Point, Pa.) which is described by the manufacturer as a partially gelatinized starch, may be used.

Extended release periods of the α₂-adrenergic receptor agonist may be provided by manipulation of the hydrophilic matrix or manipulation of the hydrophilic matrix and a release retardant. By way of example, an eight hour release period for the α₂-adrenergic receptor agonist may be provided using a hydrophilic matrix comprising Methocel® E4M which has a hydroxypropoxyl substitution of from about 7 to about 12 weight percent, a methoxyl substitution of from about 28 to about 30 weight percent, a number average molecular weight of about 86,000, a 2% aqueous solution of viscosity of about 4000 cps and 95% by weight may pass through a 100 mesh screen. By way of example, a twelve hour release period for the α₂-adrenergic receptor agonist may be provided using a hydrophilic matrix comprising Methocel® K100M, which has a hydroxypropoxyl substitution of from about 7 to about 12 weight percent, a methoxyl substitution of from about 19 to about 24 weight percent, a number average molecular weight of about 246,000, a 2% aqueous solution of viscosity of about 100,000 cps and at least 90% by weight may pass through a 100 mesh screen. By way of example, up to a twenty four hour release period for the α₂-adrenergic receptor agonist may be provided using a hydrophilic matrix comprising, for example, Methocel®, and a release retardant.

The formulation disclosed may also optionally comprise pharmaceutically acceptable formulating agents in order to promote the manufacture, compressibility, appearance and taste of the formulation. These formulating agents comprise, for example, diluents or fillers, glidants, binding agents, granulating agents, anti-caking agents, lubricants, flavors, dyes and preservatives. For example, the formulation may contain other pharmacologically-acceptable excipients for modifying or maintaining the pH, osmolarity, viscosity, clarity, color, sterility, stability, rate of dissolution, taste or odor of the formulation. The formulation may contain still other pharmacologically-acceptable excipients for modifying or maintaining the stability of one or more compounds of the composition. Such excipients are those substances usually and customarily employed to formulate dosages for administration in either unit dose or multi-dose form. The formulation herein described may be a solid oral dosage form. The solid oral dosage form is generally a tablet, capsule or gelcap. Among the optional formulating agents that further may be comprised in the matrix formulation may include, for example polyvidone; acacia gum; gelatin; alginic acid, sodium and calcium alginate; ethylcellulose; glidants such as colloidal silica, or talc; lubricants such as magnesium stearate and/or palmitate, calcium stearate, stearic acid, and polyethylene glycol.

The method can also include co-administering a therapeutically effective amount of a compound or formulation described herein and at least one other additional therapeutic agent. The composition may be co-formulated or administered with one or more additional therapeutic agents. Any therapeutic agent that is typically used in the treatment, prevention, and reduction of adrenergic dysregulation may also be administered or co-formulated with the formulations herein disclosed. The additional therapeutic agents may be administered within (either before or after) 14 days, 7 days, 24 hours, 12 hours, 1 hour, or simultaneously with the composition and/or formulations herein disclosed. Any suitable additional therapeutic agent may be co-formulated with the composition herein described or administered to the mammal being treated with this composition at concentrations known to be effective for these agents. The formulation with or without the additional agents may be administered orally or parenterally by injection, although other effective administration forms, such as intra-articular injection, intradermal injection, inhalant mists, transdermal iontophoresis or suppositories are also envisioned. The compounds and pharmaceutical formulations described herein may be used with other methods of treating and/or preventing ADHD. Other methods of treating and/or preventing ADHD include, for example, stimulants such as methylphenidate, Ritalin, Concerta, amphetamines, Adderall®, dextroamphetamines, Dexedrine®, modafinil, Provigilt, amineptine (Survector®); anti-depressants such as bupropion; nonstimulants such as Selective Norepinephrine Reuptake Inhibitors (SNRIs); tricyclic anti-depressants; Selective Serotonin Reuptake Enhancers (SSREs) such as tianeptine (Stablon®), bupropion (Wellbutrin®); and combinations thereof. The compounds and pharmaceutical formulations described herein may be used with known methods for treating hypertension, such as: ACE inhibitors such as captopril, enalapril, fosinopril (Monopril®), lisinopril (Zestril®), quinapril, ramipril (Altace®); angiotensin II receptor antagonists: e.g., irbesartan (Avapro®), losartan (Cozaar®), valsartan (Diovan®), candesartan (Atacand®); alpha blockers such as doxazosin, prazosin, or terazosin; beta blockers such as atenolol, labetalol, metoprolol (Lopressor®, Toprol-XL®); calcium channel blockers such as amlodipine (Norvasc®), diltiazem, verapamil; diuretics, such as bendroflumethiazide, chlortalidone, hydrochlorothiazide; and combinations thereof.

Methods of diagnosing and monitoring the presence or change of adrenergic dysregulation condition are generally known. To assess whether the formulations disclosed herein are useful to treat, reduce, or prevent an adrenergic dysregulation condition, any method known in the art may be used. For example, a medically desirable result for an ADHD or hypertension condition may be a reduction of impulsiveness or blood pressure, respectively. ADHD or hypertension may be diagnosed and/or monitored, for example, by physical examination of the subject before, during and after administration of the herein disclosed formulations.

The following examples describe embodiments of the invention. It will be appreciated that the amount of the agonist and its ratio with the components of the hydrophilic matrix and release-retardant with or without additional agents required for use in the treatment or prevention of an adrenergic dysregulation condition and its related complications will vary within wide limits and may be adjusted to the individual requirements of a particular subject. Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements (e.g., weights).

### EXAMPLES

The following examples are not intended to be limiting.

### Example 1

### Tablet Preparation

A preblend was prepared as follows: API (clonidine HCl, USP; Spectrum Chemical, New Brunswick, N.J.); hydroxypropyl methylcellulose (Hypromellose, USP; Methocel® K100M Premium, Dow Chemical), lactose monohydrate NF 316 Fast Flow® (Formost Farms, Wis.) pre-screened through 20 mesh was used (Tablets 1 and 2), the lactose carrier, were mixed in a V-blender, and then collected.

The pre-blend from above was combined with pre-screened partially pregelatinized starch, NF (Starch 1500®; Colorcon, PA); sodium lauryl sulfate (Spectrum Chemical, NJ) and colloidal silicon dioxide (Cab-O-Sil® M-5P; Cabot, Mass.) into a 2 qt. V-blender; and mixed for about 8 minutes; followed by a charge of pre-screened magnesium stearate and further mixing for 3 minutes. The powder was pelletized using a Fette 1200i Tablet Press to provide Tablet 1. The lactose carrier may be added before or after dry compaction of the powdered blend depending on the particular kind and particle size of the lactose. In a similar manner, additional Tablets 2-4 were prepared, and their compositions are summarized in Table 1. Tablet 5 was also prepared and its composition is summarized in Table 1.

The tablets may be film coated with art-known film coating compositions. The coating may be applied to improve the aspect and/or the taste of the tablets and the ease with which they may be swallowed. Coating the tablets may improve stability and shelf-life. Suitable coating formulations comprise a film-forming polymer such as, for example, hydroxypropyl methylcellulose, e.g. hypromellose 2910, a plasticizer such as, for example, a glycol, e.g. propylene glycol or polyethylene glycol, an opacifier, such as, for example, titanium dioxide, and a film smoothener, such as, for example, talc. Suitable coating solvents are water as well as organic solvents. Examples of organic solvents are alcohols, e.g. ethanol or isopropanol, ketones, e.g. acetone, or halogenated hydrocarbons, e.g methylene chloride. Optionally, the coating may contain a therapeutically effective amount of one or more API's to provide for an immediate release of the API(s) and thus for an immediate relief of the symptoms treated by the API(s). An ethylcellulose coating, such as Surelease® (Colorcon, PA) may be applied to the tablets in a pan coater or a fluidized bed coater.

TABLE 1

| | |
|---|---|
| | Wgt. |
| wt % of | (mg)/ |
| Formula | Tablet |

Pre-blend

| | |
|---|---|
| Clonidine HCl, USP | 0.1% |
| Lactose Monohydrate, NF (316 Fast Flow ®, | 44.1% |
| modified spray-dried) | |
| HPMC, USP (Methocel ® K100M Premium CR, | 55.8% |
| Dow) | |
| TOTAL | 100.0% |

Tablet 1

| | | |
|---|---|---|
| Clonidine HCl, USP | 0.07% | 0.1 |
| Lactose Monohydrate, NF (316 Fast Flow ®, | 64.0% | 96.0 |
| modified spray-dried) | | |
| HPMC, USP (Methocel ® K100M Premium CR, | 35.0% | 52.5 |
| Dow) | | |
| Colloidal Silicon Dioxide, NF (Cab-O-Sil ® | 0.2% | 0.3 |
| M-5P) | | |
| Magnesium Stearate, NF | 0.73% | 1.1 |
| TOTAL | 100.0% | 150.0 |

Tablet 2

| | | |
|---|---|---|
| Pre-blend | 68.1% | 81.7 |
| Partially Pregelatinized Starch, NF (Starch | 30.7% | 36.9 |
| 1500 ®) | | |
| Colloidal Silicon Dioxide, NF (Cab-0-Sil ® | 0.20% | 0.2 |
| M-5P) | | |
| Magnesium Stearate, NF | 1.0% | 1.2 |
| TOTAL | 100.0% | 120.0 |

Tablet 3

| | | |
|---|---|---|
| Pre-blend | 68.1% | 81.7 |
| Partially Pregelatinized Starch, NF (Starch | 25.7% | 30.9 |
| 1500 ®) | | |
| Sodium lauryl sulfate (SLS) | 5.0% | 6.0 |
| Colloidal Silicon Dioxide, NF (Cab-O-Sil ® | 0.2% | 0.2 |
| M-5P) | | |
| Magnesium Stearate, NF | 1.0% | 1.2 |
| TOTAL | 100.0% | 120.0 |

Tablet 4

| | | |
|---|---|---|
| Clonidine HCl, USP | 0.08% | 0.1 |
| Sodium lauryl sulfate (SLS) | 1.7% | 2.0 |
| Lactose Monohydrate, NF (316 Fast Flow ®, | 30.0% | 36.0 |
| modified spray-dried) | | |
| HPMC, Type 2208, USP | 38.0% | 45.6 |
| (Methocel ® K100M Premium CR) | | |
| Partially Pregelatinized Starch, NF (Starch | 29.1% | 34.9 |
| 1500 ®) | | |
| Colloidal Silicon Dioxide, NF (Cab-O-Sil ® | 0.2% | 0.2 |
| M-5P) | | |
| Magnesium Stearate, NF | 1.0% | 1.2 |
| TOTAL | 100.0% | 120.0 |

Tablet 5

| | | |
|---|---|---|
| Clonidine HCl, USP (SIMS) | 0.333% | 0.4 |
| Sodium Lauryl Sulfate (SLS), NF (Spectrum) | 5.933% | 7.00 |
| Lactose Monohydrate, NF (316 Fast Flow ®, | 27.500% | 33.00 |
| modified | | |
| spray-dried) | | |
| Hypromellose, Type 2208, USP (Methocel ® | 38.000% | 45.60 |
| K100M | | |
| DC Grade) | | |
| Partially Pregelatinized Starch, NF (Starch | 27.383 | 32.86 |
| 1500 ®) | | |
| Colloidal Silicon Dioxide, NF (Cab-O-Sil ® | 0.200% | 0.24 |
| M-5P) | | |
| Magnesium Stearate, NF | 0.750% | 0.90 |
| TOTAL | 100.00% | 120.00 |

### Example 2

### Active Release Profile

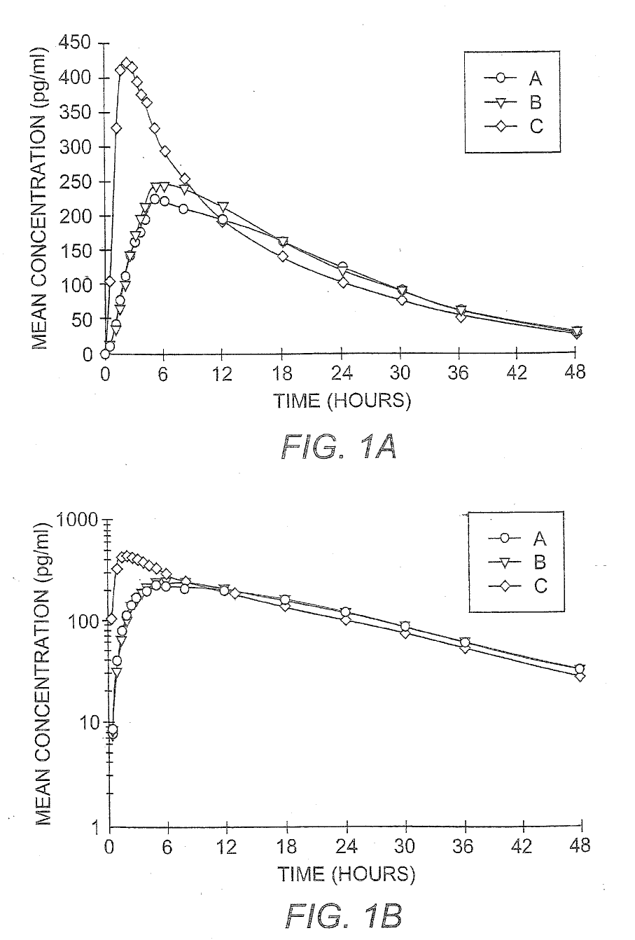

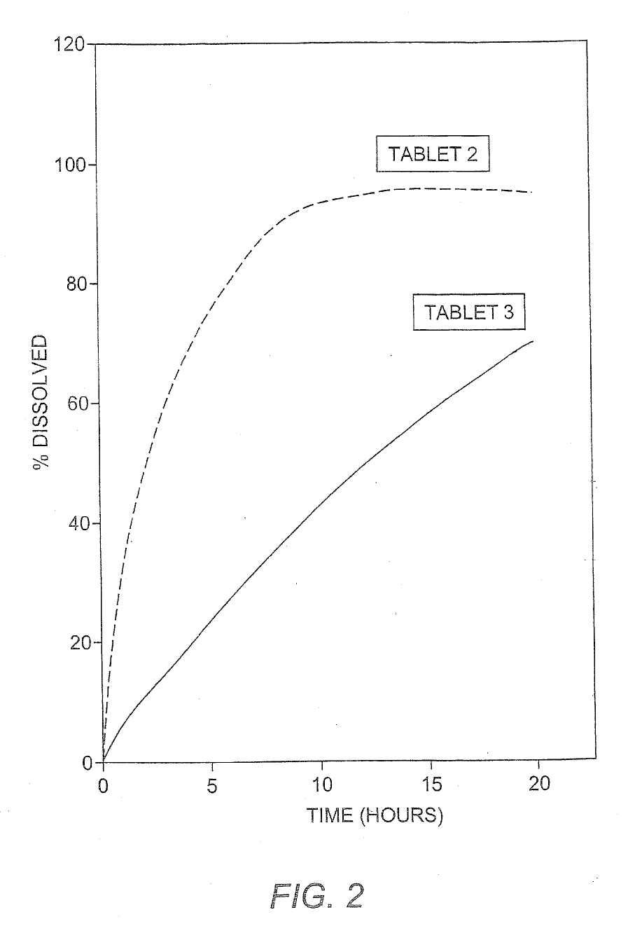

Experimental results of the dissolution of clonidine from the Tablets 2 and 3 using the USP paddle method (500 mL, 50 RPM) in a pH 2 medium are depicted in FIG. 1. The release profile is expressed as the % clonidine dissolved from the medium as a function of time. The extended release profiles of clonidine from the hydrophilic matrix with and without release-retardant are shown graphically. As shown in FIG. 1, Tablet 3, with release retardant provides a zero- to first-order release profile of clonidine as compared to Tablet 2, which is absent the release-retardant.

### Example 3

### Clinical Study of Single Dose Pharmacokinetics

During each of three treatment periods, subjects received one of the following treatments in a randomized order: CLONICEL® (clonidine HCl sustained release) 0.1 mg while fasting. CLONICEL® 0.1 mg following a standardized meal and Catapres (clonidine HCl immediate release) 0.1 mg while fasting. Blood samples for the measurement of plasma clonidine were collected pre-dose and for 48 hours following dosing. A minimum washout period of seven days separated dose administrations. A total of 15 healthy study subjects, male and female, were enrolled in the study.

After the administration of CLONICEL®, maximum clonidine concentrations are lower and occur at later times relative to clonidine concentrations after the administration of Catapres. In the present study, the Cₘₐₓ after administration of CLONICEL®-fasted was approximately 50% of Catapres Cₘₐₓ values (235±34.7 pg/mL vs. 443±59.6 pg/mL). The mean time to reach maximum concentration (Tₘₐₓ) as also longer, 6.80 hours, for CLONICEL®-fasted when compared to Catapres, which was 2.07 hours. Mean estimates of the apparent half-life of clonidine after the administration of CLONICEL®-fasted and Catapres are similar, 12.67 hours and 12.52 hours, respectively. For comparisons using an ANOVA model, the 90% confidence interval for comparing the maximum exposure of clonidine, based on ln(Cₘₐₓ), after CLONICEL®-fasted vs. Catapres is not within 80% to 125% limits. However, the 90% confidence intervals for comparing total systemic exposure, based on ln(AUCₗₐₛₜ) and ln(AUC_{inf}), are within the 80% to 125% limits, indicating the total systemic exposure to clonidine is similar after the administration of CLONICEL®-fasted and Catapres. Clonidine plasma concentration-time profiles are similar after the administration of CLONICEL® under fasted and fed conditions. In the present study, Tₘₐₓ values were 6.80 hours (fasted) and 6.50 hours (fed) and clonidine concentrations were comparable for administration under each condition. Food had no effect on the elimination half-life of CLONICEL® (12.67 hours-fasted vs.-12.65 hours-fed). The clonidine Cₘₐₓ AUCₗₐₛₜ, and AUC_{inf} ratios (fed vs. fasted) are within the 90% confidence intervals of 80% to 125%, indicating that food does not have a significant effect on either the rate or extent of absorption of clonidine from the CLONICEL® formulation. Overall, the plasma-concentration time profile of CLONICEL® was delayed and more sustained when compared to Catapres under fasted conditions and was unaffected by the presence of food. Data are shown in Tables 2, 3 and 4.

TABLE 2
Pharmacokinetic Parameters of Clonidine

| | Treatment A: | | | | Treatment B: | | | | Treatment C: | | | |
|---|---|---|---|---|---|---|---|---|---|---|---|---|
| | CLONICEL-Fasted | | | | CLONICEL-Fed | | | | CATAPRES-Fasted | | | |
| Parameter | n | Mean | SD | CV % | n | Mean | SD | CV % | n | Mean | SD | CV % |
| Tₘₐₓ (hr) | 15 | 6.80 | 3.61 | 53.05 | 14 | 6.50 | 1.23 | 18.88 | 15 | 2.07 | 0.50 | 23.96 |
| Cₘₐₓ (pg/mL) | 15 | 235 | 34.7 | 14.76 | 14 | 258 | 33.3 | 12.89 | 15 | 443 | 59.6 | 13.45 |
| AUCₗₐₛₜ (hr * pg/mL) | 15 | 5790 | 1167 | 20.16 | 14 | 5985 | 1112 | 18.57 | 15 | 6698 | 1415 | 21.12 |
| AUC_{inf} (hr * pg/mL) | 15 | 6505 | 1728 | 26.56 | 14 | 6729 | 1650 | 24.52 | 15 | 7313 | 1812 | 24.78 |
| AUC_{Extrap} (%) | 15 | 9.95 | 5.88 | 59.09 | 14 | 9.98 | 5.75 | 57.61 | 15 | 7.66 | 4.62 | 60.35 |
| λₓ (hr⁻¹) | 15 | 0.0585 | 0.0142 | 24.23 | 14 | 0.0579 | 0.0126 | 21.76 | 15 | 0.0584 | 0.0134 | 22.95 |
| T_{1/2} (h/r) | 15 | 12.67 | 3.76 | 29.66 | 14 | 12.65 | 3.56 | 28.12 | 15 | 12.52 | 3.11 | 24.83 |
| Tₗₐₛₜ (hr) | 15 | 48.01 | 0.03 | 0.06 | 14 | 47.16 | 3.21 | 6.81 | 15 | 48.00 | 0.00 | 0.00 |
| Cₗₐₛₜ (pg/mL) | 15 | 34.6 | 19.2 | 55.48 | 14 | 36.3 | 18.6 | 51.30 | 15 | 30.6 | 18.3 | 59.69 |

TABLE 3
Statistical Analysis of the Log-transformed Systemic Exposure Parameters of
CLONICEL ®-fasted (Treatment A) vs. Catapres-fasted (Treatment C).

| Dependent | Geometric Mean^{a} | | Ratio (%)^{b} | 90% CT^{c} | | | ANOVA |
|---|---|---|---|---|---|---|---|
| Variable | Test | Ref | (Test/Ref) | Lower | Upper | Power | CV % |
| ln (Cₘₐₓ) | 232.6308 | 439.5037 | 52.93 | 50.26 | 55.74 | 1.0000 | 7.96 |
| ln (AUCₗₐₛₜ) | 5690.0446 | 6573.2536 | 86.56 | 81.51 | 91.93 | 0.9998 | 9.26 |
| ln (AUC_{inf}) | 6332.2870 | 7126.9267 | 88.85 | 83.04 | 95.06 | 0.9993 | 10.42 |

TABLE 4
Statistical Analysis of the Log-transformed Systemic Exposure Parameters of
CLONICEL ®-fed (Treatment B) vs. Catapres-fasted (Treatment A).

| Dependent | Geometric Mean^{a} | | Ratio (%)^{b} | 90% CT^{c} | | | ANOVA |
|---|---|---|---|---|---|---|---|
| Variable | Test | Ref | (Test/Ref) | Lower | Upper | Power | CV % |
| ln (Cₘₐₓ) | 255.3212 | 232.5085 | 109.81 | 104.21 | 115.71 | 0.9999 | 7.68 |
| ln (AUCₗₐₛₜ) | 5846.4142 | 5677.2826 | 102.98 | 96.13 | 110.32 | 0.9990 | 10.11 |
| ln (AUC_{inf}) | 6495.4962 | 6322.5606 | 102.74 | 94.14 | 112.12 | 0.9912 | 12.86 |

### Example 4

### Clinical Study of Steady-State Plasma Concentrations with Twelve Hour Dosing

A 4-week (28 days), multi-center, double-blind, randomized, parallel group study of the steady-state pharmacokinetics and pharmacodynamics of three oral dosing regimens of CLONICEL®: 0.2, 0.4, and 0.6 mg/day was conducted. The doses in this study were chosen based on the recommended usual oral daily dose range for clonidine prescribed for hypertension and the expectation that the chosen doses would provide the range of plasma clonidine concentrations associated with efficacy in the treatment of hypertension (0.2 to 2.0 ng/mL). All doses were administered on a divided dose schedule, i.e., 0.1, 0.2, and 0.3 mg b.i.d., with 12 hours separating the doses. A total of 40 patients were projected for enrollment to achieve a minimum of 36 evaluable patients (12 per treatment arm) randomly assigned to one of the three treatment groups. Prior to random assignment to treatment, patients underwent a 2-week washout period of current antihypertensive medications. For the treatment period, an escalating titration schedule was implemented to achieve the assigned target dose. Ambulatory blood pressure monitoring (ABPM), using an appropriate monitor, was performed at Baseline prior to dosing and on the last day of dosing (Day 26). Ten blood samples to measure steady-state plasma concentrations of clonidine were collected pre-dose and for a 12-hour period following the morning dose on each of Days 23 and 25. Patients were discontinued from study medication immediately after completion of the 26-day dosing period, although they were sequestered for 48 hours (Days 27 and 28) for blood pressure and safety assessments. All patients were domiciled in the research unit specifically during all study periods that required procurement of ABPM data and blood samples for PK analyses. There were 39 patients in the PK/PD Population (12 each in the 0.2 mg/day and the 0.4 mg/day groups; and 15 in the 0.6 mg/day group), and 42 patients in the Safety Population (12 in the 0.2 mg/day group; 15 each in the 0.4 mg/day and the 0.6 mg/day groups). The active was formulated into round, white, CLONICEL® 0.1 mg oral sustained release tablets containing 0.1 mg clonidine hydrochloride.

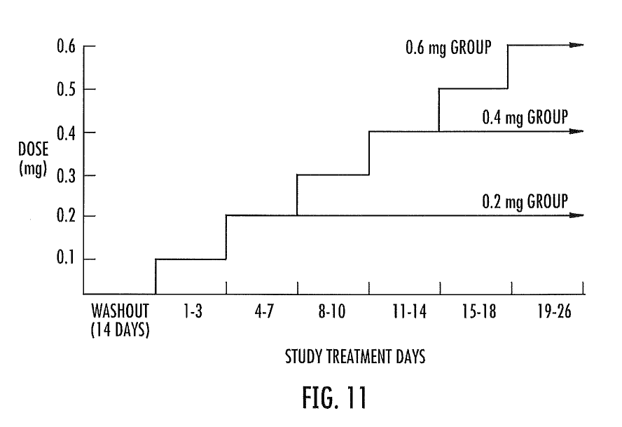

To reach steady-state plasma concentrations of the active, an escalating titration schedule was implemented. The schedule is shown in FIG. 11.

Blood samples at pre-specified intervals pre- and post study drug treatment were obtained on Days 23 and 25 for correlation of pharmacokinetics with results of ABPM obtained on Day 26-28 of the study.

Pharmacokinetic parameters for clonidine were calculated using noncompartmental analysis. Reported parameters, as defined herein, included: Maximum plasma concentrations of clonidine, observed by inspection of individual subject plots of plasma concentration versus time (Cₘₐₓ); Time (h) from dosing to Cₘₐₓ observed by inspection of individual subject plots of plasma concentration versus time (Tₘₐₓ); Minimum plasma concentrations of clonidine, observed by inspection of individual subject plots of plasma concentration versus time (Cₘᵢₙ); The average concentration during a dosing interval at steady-state. Calculated as (AUC_{0-τ}/τ(C_{avg}); the fluctuation ratio for steady-state data=Cₘₐₓ/Cₘᵢₙ; The areas under the plasma concentration-time curve during the 12-hour dosing interval at steady-state, calculated using the linear trapezoidal method (AUCτ; and The apparent oral clearance at steady-state calculated as the dose administered divided by AUCτ (CL/Fss). Individual patient pharmacokinetic analyses were conducted using actual blood sampling times and the times of dose administration. Concentration-time data that were below the limit of quantification (BLQ) were treated as zero (0.00 pg/mL) for calculation of descriptive statistics. In the pharmacokinetic analysis, BLQ concentrations were treated as zero from time-zero up to the time at which the first quantifiable concentration was observed; embedded and/or terminal BLQ concentrations were treated as “missing”. Non-compartmental pharmacokinetic parameters were calculated from plasma concentrations of clonidine on Days 23 and 25 using WinNonlin® version 5.2 (Pharsight Corp). Since CLONICEL® was administered at fixed doses, independent of body weight or size, CL/F values were normalized to body weight on a per kg basis. All derived pharmacokinetic parameters and plasma concentrations at each scheduled assessment time point were summarized with descriptive statistics (mean, standard error of the mean, standard deviation, coefficient of variation, median, range and number of observations). Graphical displays of individual subject and mean (for a given dosage level) plasma concentration versus time data were also generated.

Initial assessment of pharmacodynamic data (ABPM measurements) evaluated the effects of CLONICEL® in producing decreases in mean systolic and diastolic blood pressures across all three treatment groups. Mean baseline ABPM data for the three treatment groups were compared with mean data obtained on Day 26 (last day of treatment) and on Days 27 and 28. A more detailed PK/PD analysis was conducted by using the individual patient ABPM data to compare the blood pressure profile at Baseline and on Day 26 of treatment. The daytime (0- to 12-hour) intervals were used in the analysis, since pharmacokinetic data were collected on Day 25, over the daytime 12-hour dosing interval at steady state. In order to investigate the maintenance of effect at the tail end of the inter-dosing interval, differences between the SBP, DBP, and HR values at Baseline and the last two hours in the inter-dosing interval (Hours 11 and 12) on Day 26 were calculated for each patient. Paired t-tests were performed to test the significance of these differences between Baseline and the last two measuring times. The approach taken to quantify the effects of clonidine on blood pressure was to calculate the difference between the areas under the BP vs. time curves (AUC_{BP}) at Baseline and on Day 26. These values were used to relate the pharmacodynamic effect to dose, as well as to conduct the PK/PD analyses. Relationships between the pharmacodynamic effects and pharmacokinetic parameters including AUCτ, Cₘₐₓ and Cₘᵢₙ, were evaluated using the sigmoidal Eₘₐₓ model as follows: E=(Eₘₐₓ·C^{γ})/(C^{γ}+EC₅₀^{γ}), where E is the observed magnitude of the pharmacological effect at a given concentration; C or AUC is the drug concentration or AUC producing the pharmacological effect; Eₘₐₓ is the estimated, maximal pharmacological effect; EC₅₀ is the concentration at which the effect is 50% of the maximal effect; EC₉₀ is the concentration at which the effect is 90% of the maximal effect; and γ is the Shape factor (steepness of slope) for the E vs. C relationship.

### Analysis of Pharmacokinetic Data

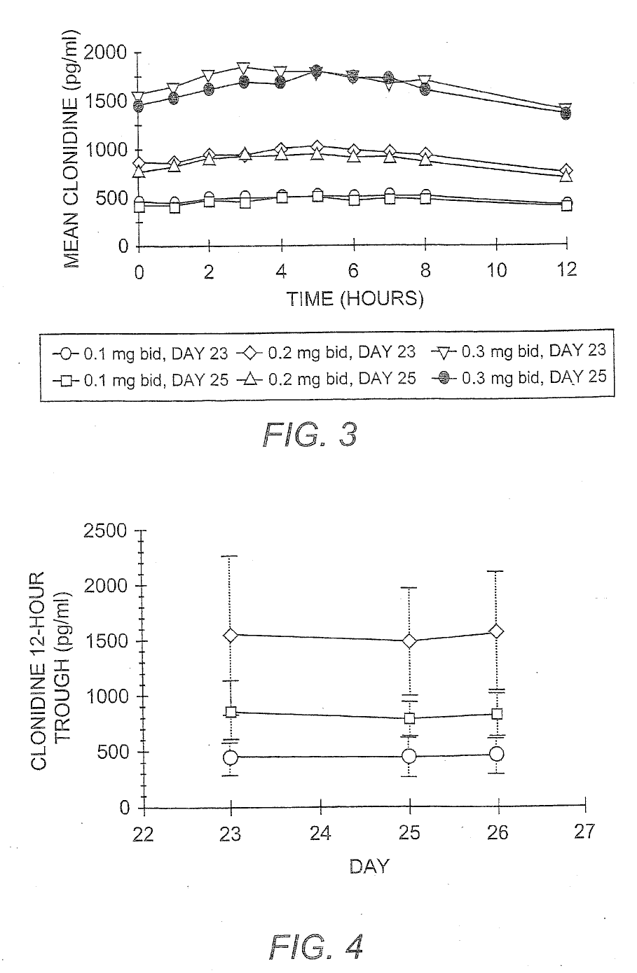

A total of 21 blood samples were collected from each patient at steady state for the assay of clonidine plasma concentrations, 10 samples on Day 23, 10 samples on Day 25, and 1 sample on Day 26. Plasma concentrations are tabulated individually by patient and plotted in FIG. 3. Average concentrations for the 3 treatment groups ranged from approximately 400 pg/mL to 1800 pg/mL. The figure shows that plasma concentrations increased proportionately with increase in dose, stayed fairly even throughout the inter-dosing interval, and were very similar between Days 23 and 25. Achievement of steady-state was confirmed by summarizing and plotting mean trough concentrations prior to the morning doses of Days 23, 25, and 26. Summary data are plotted in FIG. 4. As the figure shows, plasma clonidine concentrations were at steady-state beginning on Day 23 and throughout the sampling period. Three independent, repeated-measures ANOVA tests were performed, one for each group, to verify that the trough levels on Days 23, 25, and 26 were not statistically different. The F values and corresponding p-values for the 0.2, 0.4, and 0.6 mg groups were: F(2,22)=2.3, p=0.1237; F(2,20)=1.277, p=0.3008; F(2,28)=17.15, p=0.53, respectively. None of the ANOVA tests reached statistical significance, confirming the lack of difference between the 3 time points and the achievement of steady state.

Plasma concentrations at trough were also used to calculate intra-subject variability. It was important to investigate intra-subject variability in plasma concentration as an index of the consistency of pharmacokinetic performance between individual dosing units. The mean intra-subject coefficients of variation were very low and ranged from 10% to 12% for the three groups, thus indicating that the sustained-release formulation delivered clonidine consistently from day to day.

Steady-state noncompartmental pharmacokinetic parameters were calculated for each patient individually and summarized across treatment groups for Days 23 and 25. Table 5 shows the key parameters by treatment group and Day of treatment. Average Cₘₐₓ values ranged from 553 pg/mL for the 0.2 mg group at Day 23 to 1980 pg/mL for the 0.6 mg group at Day 23. The same pattern was evident for Cₘᵢₙ and AUC_{τ}. Tₘₐₓ averaged 4 to 5 hours at all dose levels, with an overall range for individual patients from 2 to 8 hours, although the majority (>60%) of the observed Tₘₐₓ values occurred between 4 and 6 hours. For the main derived PK parameters, the coefficients of variation (CVs) ranged from 18% to 42% with higher CVs observed at the highest dose, indicating a low inter-subject variability in pharmacokinetic exposure. Overall, there were no major differences in mean values between Days 23 and 25. The sustained-release property of the CLONICEL® formulation at steady-state was evident from the low Cₘₐₓ/Cₘᵢₙ mean ratios observed for the three treatment groups. These mean ratios averaged 1.4 to 1.5 indicating a low peak to trough fluctuation. The relationship between dose and derived PK parameters was explored by plotting Cₘₐₓ, Cₘᵢₙ, AUC, and CL/F values for Day 25 as a function of the administered dose in FIG. 4. As the figure shows, the three exposure parameters appeared to increase proportionately with the dose, and CL/F decreased slightly over the dosing range.

TABLE 5

| | | | Parameter | | | | |
|---|---|---|---|---|---|---|---|
| Treatment | | | Cₘₐₓ | Tₘₐₓ | Cₘᵢₙ | Cₘₐₓ/Cₘᵢₙ | AUCτ |
| Group | | | (pg/mL) | (h) | (pg/mL) | Ratio | (h * pg/mL) |
| 0.2 mg | Day 23 | Mean | 553 | 5.00 | 407 | 1.38 | 5867 |
| (n = 12) | | SD | 157 | 2.09 | 138 | 0.14 | 1735 |
| | Day 25 | Mean | 560 | 4.25 | 375 | 1.52 | 5627 |
| | | SD | 183 | 1.65 | 119 | 0.26 | 1594 |
| 0.4 mg | Day 23 | Mean | 1060 | 4.42 | 762 | 1.42 | 11050 |
| (n = 12) | | SD | 291 | 1.16 | 241 | 0.12 | 3196 |
| | Day 25 | Mean | 986 | 4.67 | 709 | 1.4 | 10410 |
| | | SD | 173 | 1.15 | 147 | 0.14 | 2007 |
| 0.6 mg | Day 23 | Mean | 1980 | 4.47 | 1380 | 1.44 | 20130 |
| (n = 15) | | SD | 839 | 1.81 | 568 | 0.12 | 8207 |
| | Day 25 | Mean | 1870 | 5.02 | 1320 | 1.43 | 19310 |
| | | SD | 636 | 1.52 | 451 | 0.18 | 6561 |

### Analyses of Pharmacodynamic Data

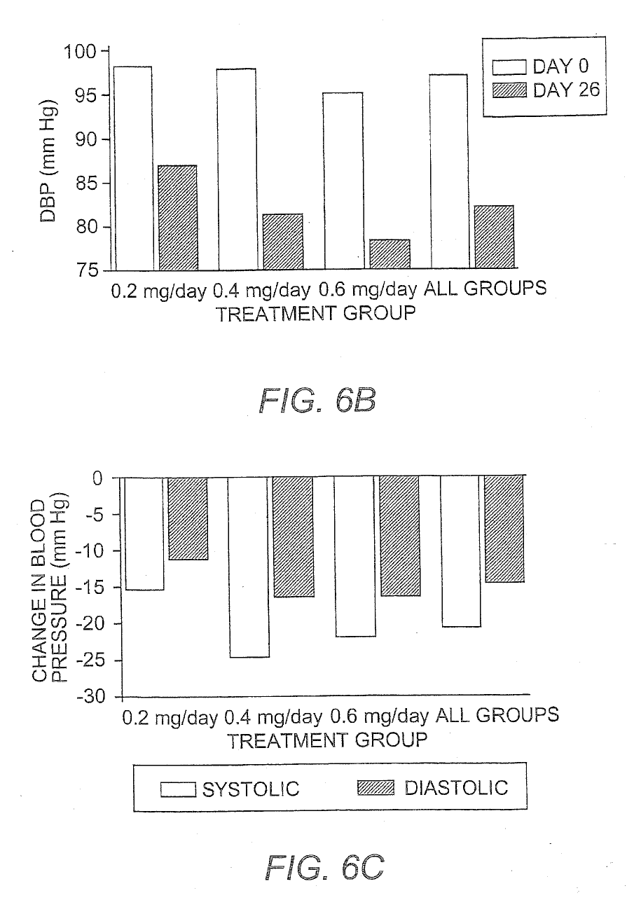

Administration of CLONICEL® at all three doses produced meaningful changes in daytime, nighttime and composite 24-hour systolic and diastolic blood pressures. The difference between Baseline and Day 26, the last day of treatment, was considered of primary importance from a pharmacodynamic perspective. Table 6 summarizes the mean daytime systolic and diastolic blood pressure observations on Baseline (Day 0) and on Days 26, 27 and 28; and FIG. 6 summarizes the data on Days 0 and 26. There was a dose-dependent reduction in mean daytime systolic and diastolic blood pressures (relative to baseline) at the 0.2 mg/day to 0.4 mg/day dose levels (15.5 mmHg and 25 mmHg reduction in mean SBP, respectively, and 11.2 mmHg and 16.6 mmHg reduction in mean DBP, respectively). However, the high dose of 0.6 mg/day did not produce further decrease in blood pressure (23.3 mmHg and 16.9 mmHg reduction in mean daytime SBP and DBP, respectively), indicating a possible plateau in blood pressure control beyond 0.4 mg/day. Forty-eight hours after abrupt cessation of study dosing, both systolic and diastolic blood pressures returned very closely to their baseline values without overshoot. The effect on blood pressure was maintained over the entire 12-hour daytime dosing interval at all doses, albeit lesser in magnitude for the 0.2 mg/day dose and between 10 and 12 hours after dosing.

TABLE 6

| Treatment | | | | | | | | |
|---|---|---|---|---|---|---|---|---|
| Group | SBP (mmHg) | | | | DBP (mmHg) | | | |
| (mg/day) | Day 0 | Day 26 | Day 27 | Day 28 | Day 0 | Day 26 | Day 27 | Day 28 |
| 0.2 (n = 12) | 146.7 | 131.2 | 135.7 | 142.9 | 98.3 | 87.1 | 89.1 | 95.6 |
| 0.4 (n = 12) | 149.1 | 124.1 | 130.0 | 143.9 | 97.9 | 81.3 | 84.3 | 94.7 |
| 0.6 (n = 15) | 147.5 | 124.2 | 134.0 | 150.4 | 95.0 | 78.1 | 83.7 | 95.5 |
| Groups | 147.7 | 126.7 | 133.3 | 146.1 | 97.0 | 81.9 | 85.5 | 95.3 |
| Combined | | | | | | | | |
| (n = 39) | | | | | | | | |

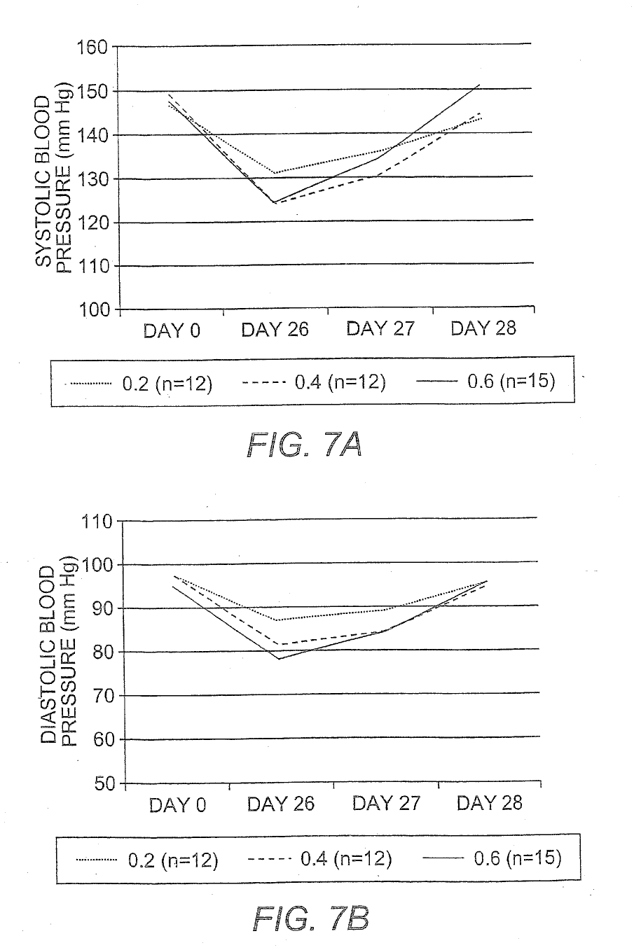

To investigate potential rebound hypertension following abrupt discontinuation of treatment with CLONICEL®, study drug was discontinued without tapering after the PM dose on Day 26. ABPM assessments were continued for 48 hours following this last dose. Table 6 and FIG. 7 summarize the mean daytime systolic and diastolic blood pressure observations at Baseline and for Days 26 to 28. As is evident from the data, both SBP and DBP daytime values gradually returned to Baseline levels over the 48 hours post-dosing without overshoot even though study medication had been withdrawn abruptly.

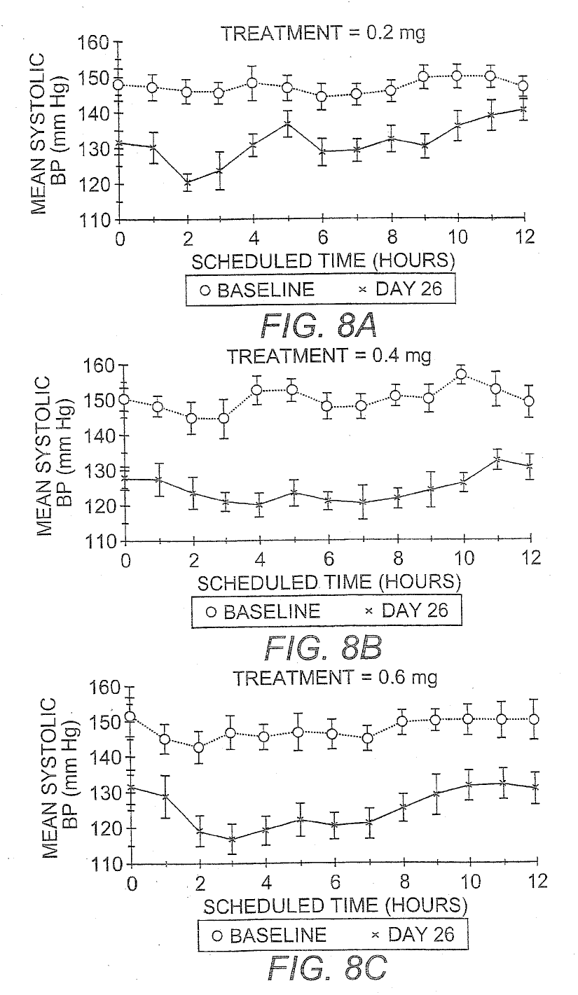

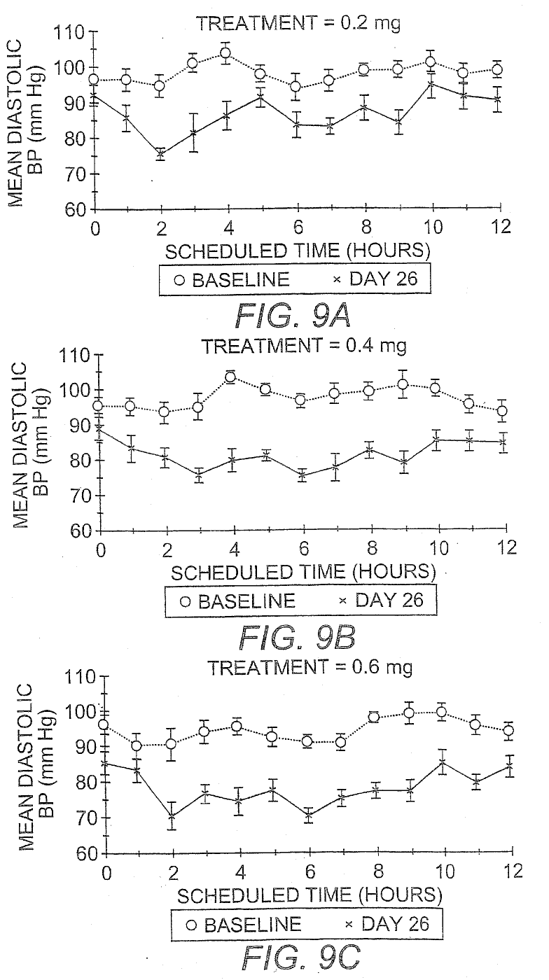

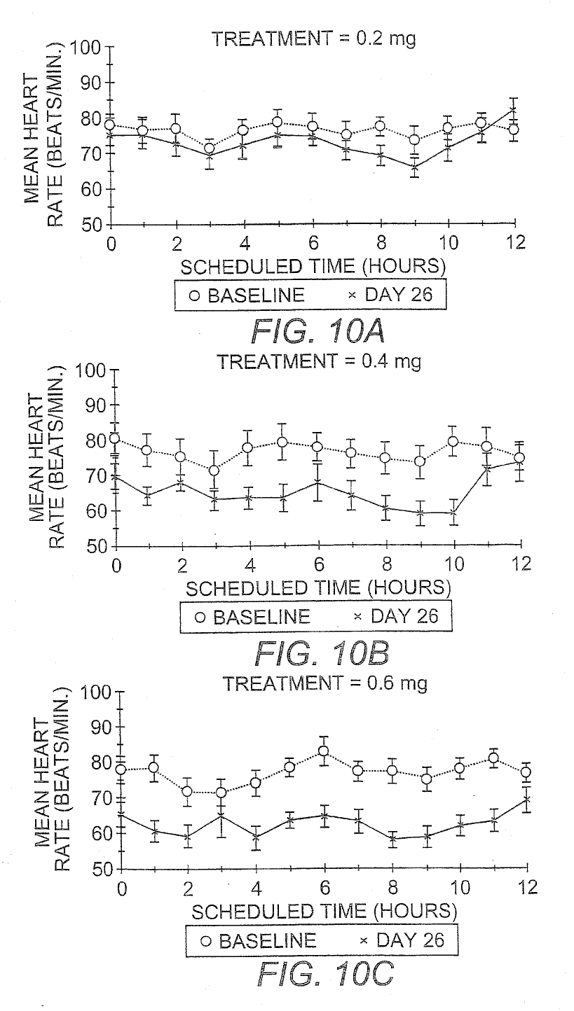

The pharmacodynamic effects of CLONICEL® throughout the inter-dosing interval were investigated by summarizing and plotting ABPM data between the morning and evening doses on Day 26, the last day of dosing. For comparison, ABPM data collected at Baseline between the same time points were also summarized and plotted. Decreases in blood pressure were observed for all but two patients during CLONICEL® treatment: the hypotensive effects were negligible for in one patient (0.2 mg/day treatment group) and absent in another (0.6 mg/day treatment group). Mean pharmacodynamic effect versus time data are plotted in FIGS. 8, 9 and 10. As observed in previous summaries, the magnitude of the effect of the morning dose on blood pressure in the 0.2 mg group appeared to be less than that elicited by the higher doses. Similar effects on blood pressure and heart rate were observed in the inter-dosing interval for the 0.4 and 0.6 mg groups. The duration of the effect on blood pressure was maintained over the entire 12-hour daytime dosing interval at the higher doses, albeit lesser in magnitude between 10 and 12 hours after dosing. To investigate the maintenance of effect at the tail end of the inter-dosing interval, differences between the SBP. DBP, and HR values at Baseline and the last two hours in the inter-dosing interval (Hours 11 and 12) on Day 26 were calculated for each patient. Table 7 summarizes the mean differences by treatment group and presents paired t-tests of the significance of these differences between Baseline and the last two measuring times. As the table shows, consistent statistically significant differences were maintained at the last 2 measuring times for SBP and DBP at the higher two dosing groups, but were more intermittent for the 0.2 mg/day group. With the exception of the difference between Hour 11 and Baseline at the 0.6 mg group, there were no statistically significant differences for HR at the last two measuring times.

TABLE 7

| | | Hour 11 | | Hour 12 | |
|---|---|---|---|---|---|
| | Treatment | mean-diff | | mean-diff | |
| Parameter | Group | (mmHg) | p-value | (mmHg) | p-value |
| Systolic | 0.2 mg | 10.82 | 0.0308 | 6.50 | 0.0676 |
| Blood | 0.4 mg | 21.91 | 0.0023 | 18.25 | 0.0021 |
| Pressure | 0.6 mg | 19.57 | 0.0053 | 19.33 | 0.0044 |
| Diastolic | 0.2 mg | 6.273 | 0.1037 | 8.500 | 0.0120 |
| Blood | 0.4 mg | 10.64 | 0.0052 | 9.000 | 0.0070 |
| Pressure | 0.6 mg | 17.00 | 0.0003 | 19.33 | 0.0044 |
| Heart Rate | 0.2 mg | 2.545 | 0.4468 | −5.333 | 0.0762 |
| | 0.4 mg | 7.727 | 0.1052 | 12.50 | 0.7731 |
| | 0.6 mg | 18.14 | 0.0029 | 7.667 | 0.1392 |

### Pharmacokinetic-Pharmacodynamic Relationships

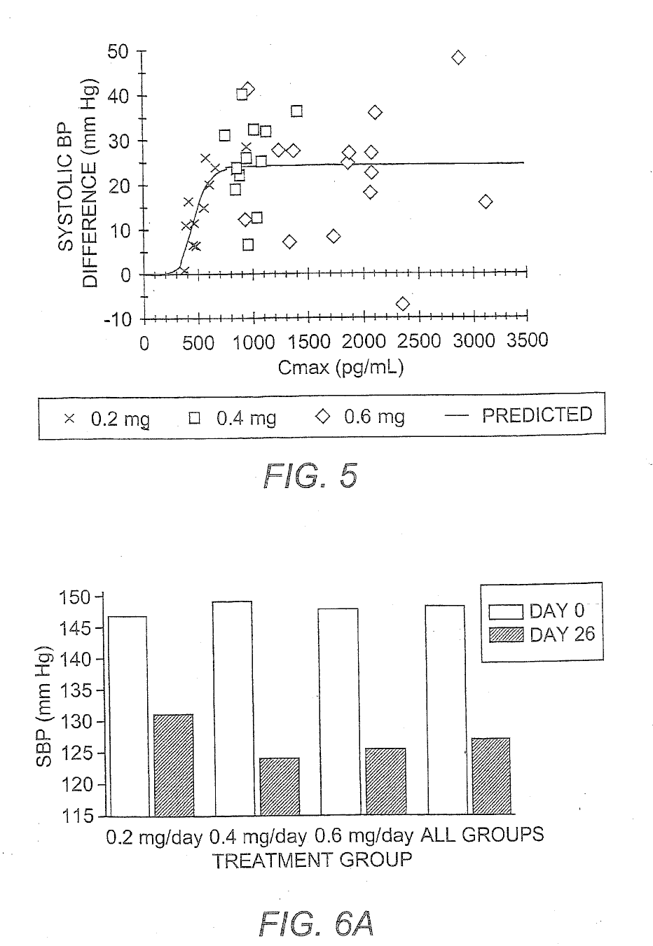

Pharmacokinetic-pharmacodynamic modeling was conducted using the blood pressure response data and the Cₘₐₓ, Cₘᵢₙ and AUCτ values at Day 25 of dosing. The sigmoidal Eₘₐₓ model (WinNonlin PD Model 105) was used with the assumption that there is no pharmacological effect at zero drug concentration. The relationships between effect and exposure were similar for changes in diastolic and systolic blood pressure for each of the 3 exposure parameters. A representative plot of the observed data, identified by CLONICEL® dose level, with the superimposed curve fit is displayed in FIG. 5. In general, the sigmoidal Eₘₐₓ model described well the relationship between blood pressure effects and clonidine concentration. The slope of the concentration-response (γ) is quite steep at the low concentrations provided by administration of the 0.2 mg daily dose of CLONICEL®. Parameter estimates for the model fits for the effects on blood pressure are summarized in Table 8. These results indicate that the clonidine concentration required to produce 50% of the maximal response on systolic blood pressure is 458 pg/mL for Cₘₐₓ and 359 pg/mL for Cₘᵢₙ Concentrations of this magnitude were consistently achieved in the 0.4 and 0.6 mg groups, but not in the 0.2 mg group. The estimated EC₉₀ for clonidine effects on systolic blood pressure indicated that Cₘₐₓ and Cₘᵢₙ concentrations of 646 and 532 pg/mL, respectively, are required. All of the patients in the 0.4 and 0.6 mg groups achieved >500 pg/mL clonidine concentrations, but based on the relationship between effect and concentration, it is apparent that little additional benefit accrues from the increase in dose beyond 0.4 mg/day. The data in Table 8 summarize the PK/PD parameters from the sigmoidal Eₘₐₓ model.

TABLE 8

| Parameter | | Systolic BP | Diastolic BP |
|---|---|---|---|
| Cₘₐₓ | Eₘₐₓ (ΔBP) | 24.3 | 16.7 |
| | EC₅₀ (pg/mL) | 458 | 431 |
| | EC₉₀ (pg/mL) | 646 | 561 |
| | Shape Factor (γ) | 6.39 | 8.29 |
| Cₘᵢₙ | Eₘₐₓ (ΔBP) | 24.4 | 16.8 |
| | EC₅₀ (pg/mL) | 359 | 341 |
| | EC₉₀ (pg/mL) | 532 | 461 |
| | Shape Factor (γ) | 5.56 | 7.31 |
| AUCτ | Eₘₐₓ (ΔBP) | 24.3 | 16.8 |
| | EC₅₀ (pg/mL) | 4702 | 4401 |
| | EC₉₀ (pg/mL) | 6692 | 5921 |
| | Shape Factor (γ) | 6.26 | 7.42 |

A sustained release profile for the CLONICEL® formulation of clonidine was confirmed by a delayed Tₘₐₓ, a dampened Cₘₐₓ, prolonged concentrations of clonidine over the 12-hour dosing interval, and low fluctuation of the plasma clonidine concentrations over the dosing interval. The low fluctuation corresponds to the narrow peak-to-trough range provided by the sustained release formulation. Low intra-subject variability in the clonidine plasma concentration-time profiles was established over two 12-hour dosing intervals at steady-state, indicates consistent delivery of clonidine by the formulation. Significant decreases in blood pressure were observed at all dose levels during treatment, with dose-related decreases at 0.2 and 0.4 mg/day but with no clinically significant additional benefit at 0.6 mg/day. Effects were maintained over the entire 12 hours in the inter-dosing interval, especially for the 0.4 and 0.6 mg/day doses. PK/PD modeling indicates that blood pressure lowering and heart rate effects were related to the steady-state AUCτ, Cₘₐₓ, and Cₘᵢₙ clonidine concentrations, with optimal effects observed at the 0.4 mg/day dose level.

### Example 5

### Clinical Study of Single 24 Hour Dose Pharmacokinetics

This was an open-label, single-dose, 3-treatment, 3-period, randomized, crossover study. The study was designed to (a) determine the single dose pharmacokinetics and bioavailability following a 0.4 mg dose of CloniDAY (a once a day clonidine formulation) given as a single 0.4 mg tablet (0.4 mg×1) under fasting conditions; (b) determine the effect of food on the bioavailability of clonidine following a 0.4 mg dose of CloniDAY given as a single 0.4 mg tablet (0.4 mg×1); and (c) determine the single dose pharmacokinetics and bioavailability of clonidine following a 0.4 mg dose of CloniDAY given as four 0.1 mg tablets (0.1 mg×4) under fasting conditions. The composition of CloniDAY is shown in Table 9.

TABLE 9

| | | Percentage By |
|---|---|---|
| Component | Weight (mg) | Weight |
| Sodium Lauryl Sulfate, NF | 6.00 | 5.0 |
| Lactose Monohydrate, NF | 33.00 | 27.5 |
| Hypromellose Type 2208, USP | 45.60 | 38.0 |
| (Methocel ® K100 Premium) | | |
| Partially Pregelatinized Starch, NF | 33.86 | 28.2 |
| Colloidal Silicon Dioxide, NF | 0.24 | 0.2 |
| Magnesium Stearate, NF | 0.90 | 0.75 |
| Total Tablet Weight | 120.0 | |

Eighteen healthy subjects were enrolled. Subjects who successfully completed the screening process checked into the research center the evening before first dose. Subjects who continued to meet inclusion/exclusion criteria the morning of dose were assigned a subject number, based on the order in which they successfully completed the screening process and procedures as outlined in the study protocol. Subjects were randomly assigned to a treatment sequence and received three separate administrations of study medication, one treatment per period, according to the randomization schedule. Subjects received each of the treatments listed in Table 10 during the three treatment periods.

TABLE 10

| | |
|---|---|
| Treatment A: | CloniDAY, Fasted Conditions |
| | Dose = 1 × 0.4 mg tablet |
| Treatment B: | CloniDAY, Fed Conditions |
| | Dose = 1 × 0.4 mg tablet |
| Treatment C: | CloniDAY, Fasted Conditions |
| | Dose = 4 × 0.1 mg tablets |

During each study period, 6 mL blood samples were obtained prior to each dosing and following each dose at selected times through 72 hours post-dose. A total of 51 pharmacokinetic blood samples were to be collected from each subject, 17 samples in each study period. In addition, blood was drawn and urine was collected for clinical laboratory testing at screening and study exit.

In each study period, subjects were admitted to the study unit in the evening prior to the scheduled dose. Subjects were confined to the research center during each study period until completion of the 36-hour blood collection and other study procedures. Subjects returned to the study unit for outpatient pharmacokinetic blood samples at 48 and 72 hours.

Blood samples (1×6 mL) were collected in vacutainer tubes containing K₂-EDTA as a preservative at pre-dose (0) and at 1, 2, 4, 6, 7, 8, 9, 10, 11, 12, 18, 24, 30, 36, 48, and 72 hours after dosing. Plasma samples were analyzed for clonidine using a validated LC-MS-MS procedure. The method was validated for a range of 8.00 to 1500 pg/mL for clonidine, based on the analysis of 0.500 mL of human EDTA plasma.

The following pharmacokinetic parameters were calculated: peak concentration in plasma (Cₘₐₓ), time to peak concentration (Tₘₐₓ), elimination rate constant (λ_{z}), terminal half-life (T_{1/2}), area under the concentration-time curve from time-zero to the time of the last quantifiable concentration (AUCₗₐₛₜ), and area under the plasma concentration time curve from time-zero extrapolated to infinity (AUC_{inf}).

Analysis of variance (ANOVA) and the Schuirmann's two one-sided t-test procedures at the 5% significance level were applied to the log-transformed pharmacokinetic exposure parameters, Cₘₐₓ, AUCₗₐₛₜ, and AUC_{inf}. The 90% confidence interval for the ratio of the geometric means (Test/Reference) was calculated. Bioequivalence was declared if the lower and upper confidence intervals of the log-transformed parameters were within 80% to 125%.

Data from 18 subjects were included in the pharmacokinetic and statistical analyses. Subject 8 completed one treatment period (Period 1, CloniDAY 4×0.1 mg-Fasted) and had limited data for one other treatment period (Period 2, CloniDAY 1×0.4 mg-Fasted). Concentration-time data for Subject 8 were retained in the data listing; however, only data for Period 1 were included in the pharmacokinetic analysis. Subject 16 completed only two treatment periods (Periods 1 and 3, CloniDAY 1×0.4 mg-Fasted and CloniDAY 4×0.1 mg-Fasted, respectively). Concentration-time data for Subject 16 were retained in the data listing and included in the pharmacokinetic and statistical analyses.

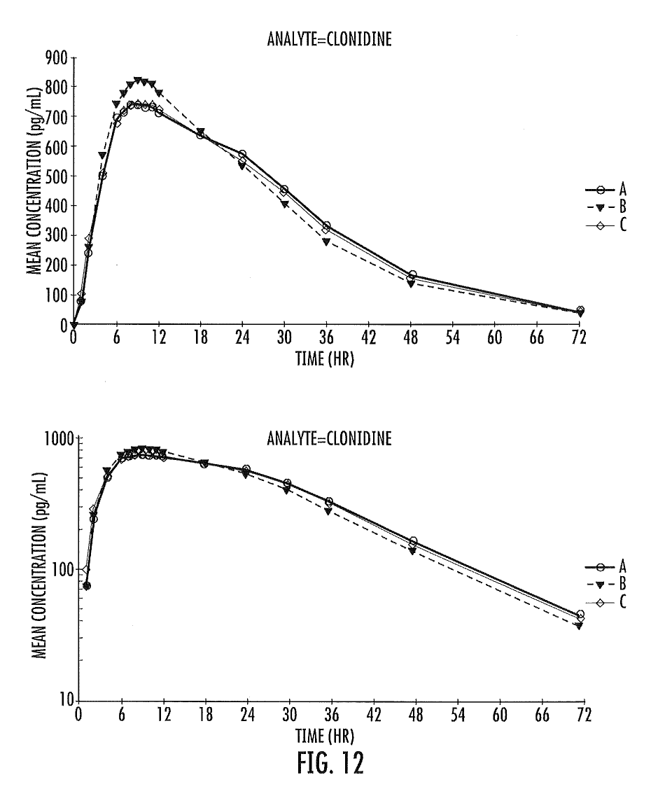

Mean concentration-time data are shown in Table 11 and FIG. 12. Results of the pharmacokinetic and statistical analyses are shown below in Tables 12 through 14.

TABLE 11
Clonidine concentration-time data after administration

| | Treatment A: | | | | Treatment B: | | | | Treatment C: | | | |
|---|---|---|---|---|---|---|---|---|---|---|---|---|
| | CloniDAY 1 × | | | | CloniDAY 1 × | | | | CloniDAY 4 × | | | |
| | 0.4 mg-Fasted | | | | 0.4 mg-Fed | | | | 0.1 mg-Fasted | | | |
| Time | | Mean | SD | CV | | Mean | SD | CV | | Mean | SD | CV |
| (hr) | n | (pg/mL) | (pg/mL) | (%) | n | (pg/mL) | (pg/mL) | (%) | n | (pg/mL) | (pg/mL) | (%) |
| 0.00 | 17 | 0.00 | 0.00 | NC | 16 | 0.00 | 0.00 | NC | 18 | 0.00 | 0.00 | NC |
| 1.00 | 17 | 76.1 | 41.9 | 55.04 | 16 | 75.4 | 50.1 | 66.47 | 17 | 101 | 50.6 | 50.04 |
| 2.00 | 17 | 240 | 104 | 43.13 | 16 | 261 | 153 | 58.76 | 18 | 290 | 72.5 | 24.97 |
| 4.00 | 17 | 498 | 88.3 | 17.72 | 16 | 571 | 173 | 30.31 | 18 | 510 | 83.9 | 16.46 |
| 6.00 | 17 | 696 | 158 | 22.74 | 16 | 745 | 196 | 26.36 | 18 | 677 | 140 | 20.73 |
| 7.00 | 17 | 715 | 176 | 24.58 | 16 | 781 | 170 | 21.70 | 18 | 722 | 160 | 22.17 |
| 8.00 | 17 | 743 | 190 | 25.60 | 16 | 810 | 155 | 19.07 | 18 | 739 | 146 | 19.73 |
| 9.00 | 17 | 741 | 191 | 25.82 | 16 | 825 | 150 | 18.16 | 18 | 744 | 144 | 19.35 |
| 10.00 | 17 | 729 | 198 | 27.09 | 16 | 820 | 148 | 18.07 | 18 | 741 | 131 | 17.70 |
| 11.00 | 17 | 734 | 205 | 27.86 | 16 | 812 | 173 | 21.26 | 18 | 743 | 137 | 18.40 |
| 12.00 | 17 | 713 | 182 | 25.50 | 16 | 782 | 151 | 19.26 | 18 | 723 | 124 | 17.09 |
| 18.00 | 17 | 637 | 168 | 26.40 | 16 | 652 | 85.0 | 13.04 | 18 | 638 | 107 | 16.73 |
| 24.00 | 17 | 577 | 168 | 29.19 | 16 | 538 | 90.2 | 16.77 | 18 | 554 | 108 | 19.52 |
| 30.00 | 17 | 457 | 144 | 31.55 | 16 | 407 | 83.0 | 20.39 | 18 | 448 | 102 | 22.86 |
| 36.00 | 17 | 334 | 114 | 34.06 | 16 | 281 | 73.1 | 26.00 | 18 | 322 | 86.2 | 26.80 |
| 48.00 | 17 | 168 | 67.2 | 40.08 | 16 | 140 | 53.7 | 38.46 | 18 | 156 | 50.4 | 32.28 |
| 72.00 | 17 | 44.4 | 24.6 | 55.38 | 16 | 37.8 | 24.2 | 64.10 | 18 | 42.9 | 21.9 | 51.06 |

Note:
Plasma samples analyzed using a bioanalytical method with a validated range 8.00 to 1500 pg/mL; concentrations reported in pg/mL to 3 significant figures; concentrations below limit of quantification set to zero (0.00 pg/mL) in the data summarization
NC = Not calculated

TABLE 12
Pharmacokinetic parameters of clonidine

| | Treatment A: | | | | Treatment B: | | | | Treatment C: | | | |
|---|---|---|---|---|---|---|---|---|---|---|---|---|
| | CloniDAY 1 × 0.4 mg- | | | | CloniDAY 1 × 0.4 mg- | | | | CloniDAY 4 × 0.1 mg- | | | |
| | Fasted | | | | Fed | | | | Fasted | | | |
| Parameter | n | Mean | SD | CV % | n | Mean | SD | CV % | n | Mean | SD | CV % |
| Tₘₐₓ (hr) | 17 | 11.35 | 5.57 | 49.04 | 16 | 9.50 | 3.88 | 40.85 | 18 | 9.89 | 2.61 | 26.39 |
| Tₘₐₓ Median* | 17 | 11.00 [6.00-24.00] | | | 16 | 9.00 [4.00-18.00] | | | 18 | 9.00 [7.00-18.00] | | |
| Cₘₐₓ (pg/mL) | 17 | 801 | 166 | 20.68 | 16 | 919 | 176 | 19.15 | 18 | 789 | 145 | 18.42 |
| AUCₗₐₛₜ | 17 | 25230 | 5867 | 23.25 | 16 | 24550 | 2581 | 10.51 | 18 | 24860 | 4242 | 17.06 |
| (hr * pg/mL) | | | | | | | | | | | | |
| AUC_{inf} | 17 | 26070 | 6113 | 23.45 | 16 | 25320 | 2850 | 11.26 | 18 | 25680 | 4539 | 17.68 |
| (hr * pg/mL) | | | | | | | | | | | | |
| AUC_{Extrap} (%) | 17 | 3.15 | 1.98 | 62.89 | 16 | 2.95 | 2.01 | 68.08 | 18 | 3.09 | 1.74 | 56.49 |
| λ_{z} (hr⁻¹) | 17 | 0.0579 | 0.0097 | 16.76 | 16 | 0.0601 | 0.0121 | 20.05 | 18 | 0.0583 | 0.0109 | 18.69 |
| T_{1/2} (hr) | 17 | 12.31 | 2.18 | 17.70 | 16 | 11.96 | 2.43 | 20.29 | 18 | 12.28 | 2.28 | 18.52 |
| Tₗₐₛₜ (hr) | 17 | 72.00 | 0.00 | 0.00 | 16 | 70.50 | 6.00 | 8.51 | 18 | 72.02 | 0.10 | 0.14 |
| Cₗₐₛₜ (pg/mL) | 17 | 44.4 | 24.6 | 55.38 | 16 | 41.3 | 22.4 | 54.19 | 18 | 42.9 | 21.9 | 51.06 |

Note:
Full precision data used in pharmacokinetic analysis
*Tₘₐₓ presented in hours (hr) as Median [Range]

TABLE 13
Statistical Analysis of the Log-Transformed Systemic Exposure Parameters of
Clonidine Comparing CloniDAY 1 × 0.4 mg-Fed (Treatment B) vs. CloniDAY
1 × 0.4 mg-Fasted (Treatment A)

| Dependent | Geometric Mean^{a} | | Ratio (%)^{b} | 90% CT^{c} | | | ANOVA |
|---|---|---|---|---|---|---|---|
| Variable | Test | Ref | (Test/Ref) | Lower | Upper | Power | CV % |
| ln (Cₘₐₓ) | 903.6363 | 778.2812 | 116.11 | 103.78 | 129.90 | 0.9481 | 18.01 |
| ln (AUCₗₐₛₜ) | 24485.1915 | 24167.4310 | 101.31 | 90.77 | 113.08 | 0.9546 | 17.62 |
| ln (AUC_{inf}) | 25233.4444 | 24981.1045 | 101.01 | 90.40 | 112.86 | 0.9518 | 17.79 |

^{a}Geometric Mean for CloniDAY 1 × 0.4 mg-Fed (Test) and CloniDAY 1 × 0.4 mg-Fasted (Ref) based on Least Squares Mean of log-transformed parameter values
^{b}Ratio (%) = Geometric Mean (Test)/Geometric Mean (Ref)
^{c}90% Confidence Interval

TABLE 14
Statistical Analysis of the Log-Transformed Systemic Exposure Parameters of
Clonidine Comparing CloniDAY 4 × 0.1 mg-Fasted (Treatment C) vs. CloniDAY
1 × 0.4 mg-Fasted (Treatment A)

| Dependent | Geometric Mean^{a} | | Ratio (%)^{b} | 90% CT^{c} | | | ANOVA |
|---|---|---|---|---|---|---|---|
| Variable | Test | Ref | (Test/Ref) | Lower | Upper | Power | CV % |
| ln (Cₘₐₓ) | 768.1445 | 781.0335 | 98.35 | 92.74 | 104.29 | 0.9999 | 9.70 |
| ln (AUCₗₐₛₜ) | 24443.4306 | 24163.0512 | 101.16 | 89.68 | 114.11 | 0.9222 | 20.07 |
| ln (AUC_{inf}) | 25251.8994 | 24965.5673 | 101.15 | 89.29 | 114.57 | 0.9073 | 20.77 |

^{a}Geometric Mean for CloniDAY 4 × 0.1 mg-Fasted (Test) and CloniDAY 1 × 0.4 mg-Fasted (Ref) based on Least Squares Mean of log-transformed parameter values
^{b}Ratio (%) = Geometric Mean (Test)/Geometric Mean (Ref)
^{c}90% Confidence Interval

### Concentration-Time Data

The first quantifiable clonidine concentrations were observed at the 1.00-hour sample time for all treatments. The highest mean plasma clonidine concentrations were 743±190 pg/mL at 8.00 hr for CloniDAY 1×0.4 mg-Fasted, 825±150 pg/mL at 9.00 hr for CloniDAY 1×0.4 mg-Fed, and 744±144 pg/mL at 9.00 hr for CloniDAY 4×0.1 mg-Fasted. Quantifiable concentrations of clonidine were observed throughout the 72-hour sampling interval for most subjects after each treatment.

### Pharmacokinetic Parameters

After the administration of CloniDAY 1×0.4 mg-Fed, maximum clonidine plasma concentrations were higher and occurred earlier in time relative to those after administration of CloniDAY 1×0.4 mg-Fasting; clonidine concentrations after CloniDAY 4×0.1 mg-Fasting were comparable to those after CloniDAY 1×0.4 mg-Fasting. Mean Cₘₐₓ values were 801±166 pg/mL after CloniDAY 1×0.4 mg-Fasting, 919±176 pg/mL after CloniDAY 1×0.4 mg-Fed, and 789±145 pg/mL after CloniDAY 4×0.1 mg-Fasting. Median [Range] Tₘₐₓ values were 11.00 hr [6.00-24.00 hr] after CloniDAY 1×0.4 mg-Fasting, 9.00 hr [4.00-18.00 hr] after CloniDAY 1×0.4 mg-Fed, and 9.00 hr [7.00-18.00 hr] after CloniDAY 4×0.1 mg-Fasting. Although differences were observed between CloniDAY 1×0.4 mg under fasting and fed conditions with regard to maximum clonidine exposure, such differences were not observed for overall systemic exposure to clonidine. Mean estimates of AUCₗₐₛₜ were comparable across all treatments in this study at 25230±5867 hr*pg/mL after CloniDAY 1×0.4 mg-Fasting, 24550±2581 hr*pg/mL after CloniDAY 1×0.4 mg-Fed, and 24860±4242 hr*pg/mL after CloniDAY 4×0.1 mg-Fasting. Mean estimates of AUC_{inf} were similar to the mean AUCₗₐₛₜ values and, on average, only approximately 3% of AUC_{inf} was based on extrapolation. Mean estimates of clonidine T_{1/2} were comparable across all treatments in this study and ranged from 11.96±2.43 hr after the administration of CloniDAY 1×0.4 mg-Fed to 12.31±2.18 hr after the administration of CloniDAY 1×0.4 mg-Fasting.

### Statistical Analysis

CloniDAY 1×0.4 mg—Fed vs. CloniDAY 1×0.4 mg—Fasted

From the ANOVA results, the 90% confidence intervals for comparing overall systemic exposure to clonidine (based on AUCₗₐₛₜ and AUC_{inf}) after the administration of CloniDAY 1×0.4 mg-Fed to that after CloniDAY 1×0.4 mg-Fasted were within the 80% to 125% range, indicating that systemic exposure to clonidine was comparable for these two treatments and that no significant food effect was observed on the extent of absorption. The geometric mean ratios (90% confidence intervals) were 101.31% (90.77, 113.08%) for AUCₗₐₛₜ and 101.01% (90.40, 112.86%) for AUC_{inf}. A significant difference was observed for peak exposure to clonidine, with an approximate 16% increase in Cₘₐₓ after CloniDAY 1×0.4 mg—Fed relative to that after CloniDAY 1×0.4 mg—Fasted; the geometric mean ratio (90% confidence interval) was 116.11% (103.78, 129.90%) for Cₘₐₓ.

CloniDAY 4×0.1 mg—Fasted vs. CloniDAY 1×0.4 mg—Fasted

From the ANOVA results, the 90% confidence intervals were within the 80% to 125% range for Cₘₐₓ, AUCₗₐₛₜ, and AUC_{inf}, indicating comparable peak and overall exposure to clonidine after CloniDAY 4×0.1 mg-Fasted and CloniDAY 1×0.4 mg-Fasted. The geometric mean ratios (90% confidence intervals) were 98.35% (92.74, 104.29%) for Cₘₐₓ, 101.16% (89.68, 114.11%) for AUCᵢₐₛₜ and 101.15% (89.29, 114.57%) for AUC_{inf}. Bioequivalence was demonstrated for these two treatments.

Based on mean estimates of Cₘₐₓ, maximum clonidine plasma concentrations after the administration of CloniDAY 1×0.4 mg-Fed were higher and occurred earlier relative to those after administration of CloniDAY 1×0.4 mg-Fasting; maximum clonidine concentrations after CloniDAY 4×0.1 mg-Fasting were comparable to those after CloniDAY 1×0.4 mg-Fasting.

Based on mean estimates of AUCₗₐₛₜ and AUC_{inf}, overall systemic exposure to clonidine was comparable after CloniDAY 1×0.4 mg-Fasting, CloniDAY 1×0.4 mg-Fed, and CloniDAY 4×0.1 mg-Fasting. The 72-hour sampling interval used in the present study captured most of the clonidine exposure, with only approximately 3% of AUC_{inf} based on extrapolation.

Mean estimates of clonidine T_{1/2} were comparable across all treatments in this study and ranged from 11.96±2.43 hr after the administration of CloniDAY 1×0.4 mg-Fed to 12.31±2.18 hr after the administration of CloniDAY 1×0.4 mg-Fasting.

The 90% confidence intervals for comparing overall systemic exposure to clonidine (based on AUCₗₐₛₜ and AUC_{inf}) after the administration of CloniDAY 1×0.4 mg-Fed to that after CloniDAY 1×0.4 mg-Fasted were within the 80% to 125% range, indicating no significant food effect on the extent of absorption. However, a significant difference was observed for peak exposure to clonidine, with an approximate 16% increase in Cₘₐₓ after CloniDAY 1×0.4 mg-Fed relative to that after CloniDAY 1×0.4 mg-Fasted and the 90% confidence interval was slightly outside of the 80% to 125% range.

The 90% confidence intervals for comparing Cₘₐₓ, AUCₗₐₛₜ, and AUC_{inf} after CloniDAY 4×0.1 mg-Fasted to those parameters after CloniDAY 1×0.4 mg-Fasted were within the 80% to 125% range, indicating comparable peak and overall exposure to clonidine. These results demonstrate that CloniDAY 4×0.1 mg-Fasted is bioequivalent to CloniDAY 1×0.4 mg-Fasted.

### Example 6

### Clinical Study of Steady-State Plasma Concentrations with 24 Hour Dosing

This was an open-label, 2-treatment, 2-period, randomized, crossover, multiple dose study. This study was designed to (a) determine the inter- and intra-subject variability of the steady-state pharmacokinetic parameters for clonidine on two separate days of a 7-day dosing regimen of CloniDAY 0.2 mg; and (b) compare the steady-state pharmacokinetic parameters for clonidine from a 7-day regimen of 0.2 mg of CloniDAY administered once daily to a 6-day regimen of Sympres (a twice a day clonidine formulation) 0.2 mg given as one 0.1 mg tablet every 12 hours. The composition of Sympres is shown in Table 15.

TABLE 15

| | | Percentage By |
|---|---|---|
| Component | Weight (mg) | Weight |
| Sodium Lauryl Sulfate, NF | 2.00 | 1.7 |
| Lactose Monohydrate, NF | 36.00 | 30.0 |
| Hypromellose Type 2208, USP | 45.60 | 38.0 |
| (Methocel ® K100 Premium) | | |
| Partially Pregelatinized Starch, NF | 35.16 | 29.3 |
| Colloidal Silicon Dioxide, NF | 0.24 | 0.2 |
| Magnesium Stearate, NF | 0.90 | 0.75 |
| Total Tablet Weight | 120.0 | |

Eighteen (18) healthy subjects were enrolled. Subjects were randomly assigned to a treatment sequence and received study medications, one treatment per period, according to the randomization schedule.

A screening period of up to 21 days preceded initiation of Period 1. Subjects who satisfied all preliminary entry criteria returned for a Baseline visit, during which eligibility was confirmed. Subjects returned to the research unit on the morning of Day 1 for the start of dosing.

On Days 1 and 2 of both periods, subjects were administered their first dose of CloniDAY or Sympres at 8:00 am (±2 hours) at the research unit. Subjects remained at the research unit for 8 hours following dosing for safety assessments before being discharged for the evening. Subjects receiving Sympres, were dispensed their evening dose of study drug prior to being discharged. On Day 2, subjects were dispensed enough study medication to last through Day 5. On the evening of Day 5, at least 10 hours prior to the Day 6 dose, subjects returned to the research unit and were confined to the research unit until after the 24-hour sample was collected on Day 6 (Sympres treatment group) or Day 7 (CloniDAY treatment group). Subjects received each of the treatments listed in Table 16 during the two treatment periods.

TABLE 16

| | |
|---|---|
| Treatment A: | Test Formulation |
| | CloniDAY |
| | Dose = 2 × 0.1 mg tablets QD |
| Treatment B: | Reference: Product |
| | Sympres |
| | Dose = 0.1 mg tablet BID |

During each study period, 6 mL blood samples were obtained during Days 6 and 7 (CloniDAY) or Day 6 (Sympres). A total of 44 pharmacokinetic blood samples were to be collected from each subject, 25 samples during the CloniDAY treatment period and 19 samples during the Sympres treatment period. In addition, blood was drawn and urine was collected for clinical laboratory testing at screening and study exit.

Blood samples (1×6 mL) were collected in vacutainer tubes containing K₂-EDTA as a preservative. Sample schedules were as follows.

CloniDay
Day 6: Pre-dose, and 1, 2, 4, 6, 7, 8, 9, 10, 11, 12, 18, and 24 hours post dose. Note: The 24-hour post dose sample collection also served as the Day 7 pre-dose sample collection.
Day 7: 1, 2, 4, 6, 7, 8, 9, 10, 11, 12, 18, and 24 hours post dose. Sympres
Day 6: Pre-dose, and 1, 2, 4, 5, 6, 7, 8, 10, 12, 13, 14, 16, 17, 18, 19, 20, 22, and 24 hours post the morning dose. The blood sample collected at 12 hours post-dose was to be collected prior to dispensing the evening dose.

Plasma samples were analyzed for clonidine using a validated LC-MS-MS procedure. The method was validated for a range 8.00 to 1500 pg/mL for clonidine, based on the analysis of 0.500 mL of human EDTA plasma.

The following pharmacokinetic parameters were calculated: peak concentration in plasma (Cₘₐₓ), minimum concentration in plasma (Cₘᵢₙ), time to peak concentration (Tₘₐₓ), elimination rate constant (λ₇), terminal half-life (T_{1/2}), area under the concentration-time curve from time-zero to 24 hours (AUC₀₋₂₄), area under the concentration-time curve from time-zero through the 24-hour dosing interval for CloniDAY or first 12-hour dosing interval for Sympres (AUC_{0-τ}), area under the concentration-time curve from time-zero to the time of the last quantifiable concentration (AUCₗₐₛₜ), total systemic clearance CLₛₛ/F, and percent fluctuation.

Attainment of steady-state was assessed using pre-dose (trough) clonidine concentrations (Day 6 pre-dose, Day 7 pre-dose, Day 7 24 hours for CloniDAY QD; Day 6 pre-dose, 12 hours, 24 hours for Sympres BID) and Tukey's multiple comparison test.

Analysis of variance (ANOVA) and the Schuirmann's two one-sided t-test procedures at the 5% significance level were applied to the log-transformed pharmacokinetic exposure parameters, Cₘₐₓ, Cₘᵢₙ, AUC_{0-τ}, (CloniDAY Day 6), and AUC₀₋₂₄ (Sympres Day 6). The 90% confidence interval for the ratio of the geometric means (Test/Reference) was calculated. Bioequivalence was declared if the lower and upper confidence intervals of the log-transformed parameters were within 80% to 125%. Intra-subject variability was estimated as the ANOVA CV % from a comparison of the pharmacokinetic parameters Cₘₐₓ, Cₘᵢₙ, and AUC_{0-τ} after the administration of CloniDAY on consecutive study days (Day 6 and Day 7).

Data from 16 subjects were included in the pharmacokinetic and statistical analyses.

Subject 113 had limited data for only one treatment period (Period 1, CloniDAY 0.2 mg-QD, Day 6). Concentration-time data for Subject 113 were retained in the data listing. However, these data were excluded from the pharmacokinetic and statistical analyses due to the crossover design of this study and the fact that this subject received only one treatment.

Subjects 108 and 110 were identified as potential outliers, having low predose concentrations of clonidine on Day 6 in the CloniDAY 0.2 mg-QD treatment groups. In addition, the variability in predose clonidine concentration across Days 6 and 7 for these two subjects was high at 79.9% and 87.9%, respectively, compared to other subject in the study (see Table 17) and a pronounced increase in concentration was noted after the in-house CloniDAY administration on Day 6; the variability in predose concentrations for all subjects dosed with CloniDAY 0.2 mg-QD ranged from 2.25% to 87.90% (Table 17). These observations suggested possible non-compliance for Subjects 108 and 110 during outpatient CloniDAY administration on. Days 3 through 5.

TABLE 17
Mean Clonidine Predose Concentrations of Individual Subjects after
Administration of CloniDAY 0.2 mg-QD (Treatment A) and Sympres
0.1 mg-BID (Treatment B) on Days 6 and 7

| | Treatment A: | | | | Treatment B: | | | |
|---|---|---|---|---|---|---|---|---|
| | CloniDAY 0.2 mg-QD | | | | Sympres 0.1 mg-BID | | | |
| | | Mean | SD | CV | | Mean | SD | CV |
| Subject # | n | (pg/mL) | (pg/mL) | (%) | n | (pg/mL) | (pg/mL) | (%) |
| 101 | 3 | 321 | 108 | 33.58 | 3 | 532 | 50.5 | 9.49 |
| 102 | 3 | 427 | 72.0 | 16.88 | 3 | 547 | 29.9 | 5.47 |
| 103 | 3 | 672 | 54.8 | 8.15 | 3 | 697 | 19.8 | 2.83 |
| 104 | 3 | 561 | 12.6 | 2.25 | 3 | 792 | 20.9 | 2.64 |
| 105 | 3 | 412 | 66.5 | 16.15 | 3 | 645 | 36.9 | 5.72 |
| 106 | 3 | 426 | 19.3 | 4.52 | 3 | 497 | 50.9 | 10.25 |
| 108 | 3 | 252 | 201 | 79.90 | 3 | 393 | 148 | 37.57 |
| 109 | 3 | 306 | 46.0 | 15.00 | 3 | 301 | 21.3 | 7.08 |
| 110 | 3 | 292 | 257 | 87.90 | 3 | 391 | 113 | 29.03 |
| 111 | 3 | 332 | 98.0 | 29.52 | 3 | 442 | 79.8 | 18.04 |
| 112 | 3 | 384 | 32.7 | 8.53 | 3 | 332 | 46.9 | 14.10 |
| 114 | 3 | 479 | 53.2 | 11.11 | 3 | 641 | 54.3 | 8.47 |
| 115 | 3 | 227 | 88.6 | 38.99 | 3 | 628 | 52.1 | 8.30 |
| 116 | 3 | 199 | 106 | 53.41 | 3 | 436 | 32.5 | 7.44 |
| 117 | 3 | 466 | 156 | 33.41 | 3 | 503 | 25.1 | 4.98 |
| 118 | 3 | 381 | 105 | 27.51 | 3 | 525 | 70.3 | 13.39 |

Note:
Plasma samples analyzed using a bioanalytical method with a validated range 8.00 to 1500 pg/mL; concentrations reported in pg/mL to 3 significant figures; concentrations below limit of quantification set to zero (0.00 pg/mL) in the data summarization

Due to the high degree of variability and increase in predose concentrations across study days for Subjects 108 and 110, the T Procedure (Grubb's test) and the Dixon's test were used to determine if there were any outliers in the CloniDAY data set. The basis of these tests was Cₘᵢₙ on Day 6 and Day 7, determined during noncompartmental pharmacokinetic analysis. The test criteria included Cₘᵢₙ on Day 6 and Day 7 (each day investigated separately), difference in Cₘᵢₙ between Day 6 and Day 7, and the ratio of Cₘᵢₙ across days (Day 6: Day 7). No subjects were formally identified as outliers in these tests, perhaps due to the wide range of Cₘᵢₙ values observed. However, the concentration-time profiles for Subjects 108 and 110 suggested that steady state conditions had not been achieved for these two subjects during the outpatient dosing and additional tests were conducted. The results of Tukey's test comparing the predose concentrations across study days (discussed below) indicated a significant difference in predose concentrations between Days 6 and 7 when all subjects were included in the analysis. After repeating Tukey's test and excluding Subjects 108 and 110, no significant differences in predose concentrations were observed, demonstrating that steady state conditions were achieved.

Since the objective of this study was to assess the pharmacokinetic profile of clonidine at steady state and due to the observation that steady state conditions may not have been achieved for Subjects 108 and 110 as well as the indication and possibility that these subjects were non-compliant with regard to the outpatient administration of CloniDAY 0.2 mg-QD, Subjects 108 and 110 were excluded from the primary pharmacokinetic and statistical analyses.

### Concentration-Time Data

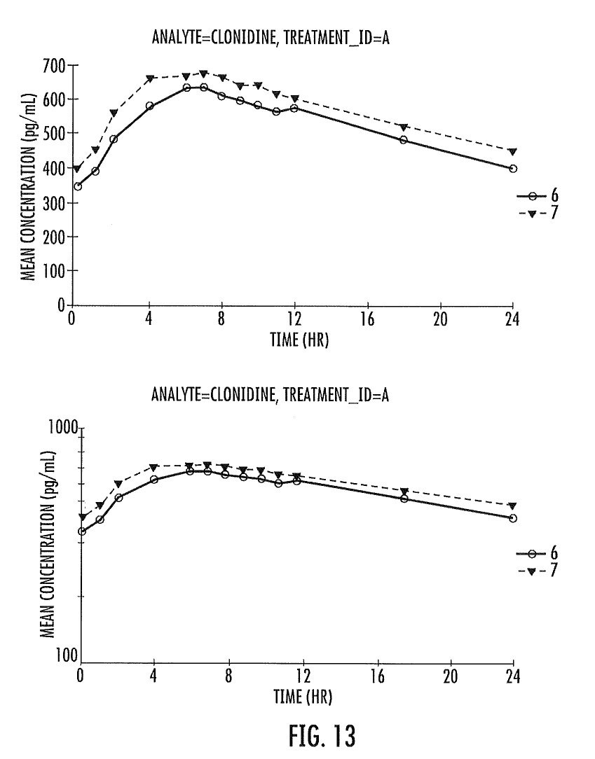

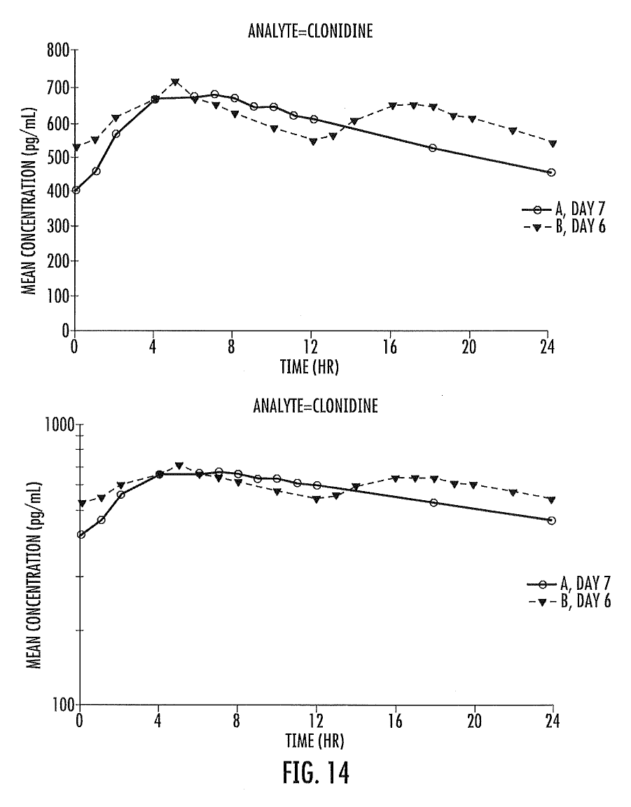

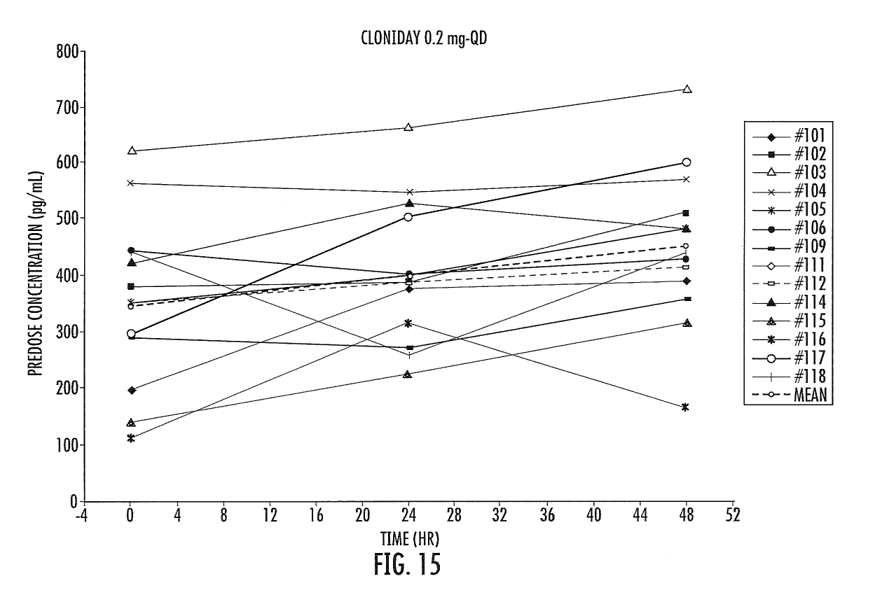

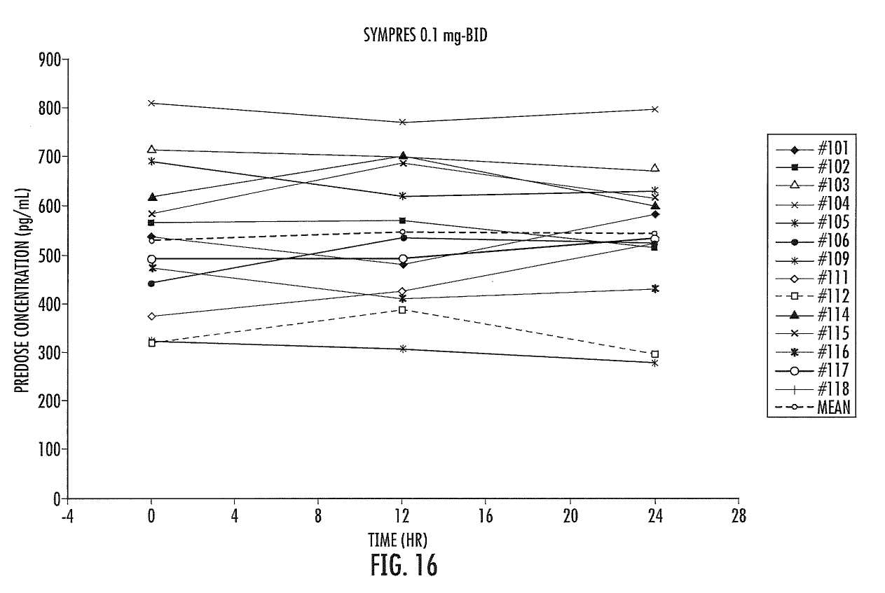

Mean concentration-time data are shown in Tables 18 and 19 and FIGS. 13 and 14. Predose concentrations during Days 6 and 7 are shown in Table 20 and FIGS. 15 and 16.

TABLE 18
Clonidine Concentration-Time Data after Administration of
CloniDAY 0.2 mg-QD (Treatment A) on Days 6 and 7 Excluding
Subjects 108 and 110

| | Treatment A: | | | | Treatment A: | | | |
|---|---|---|---|---|---|---|---|---|
| | CloniDAY 0.2 mg-QD | | | | CloniDAY 0.2 mg-QD | | | |
| | Day 6 | | | | Day 7 | | | |
| Time | | Mean | SD | CV | | Mean | SD | CV |
| (hr) | n | (pg/mL) | (pg/mL) | (%) | n | (pg/mL) | (pg/mL) | (%) |
| 0.00 | 14 | 346 | 148 | 42.86 | 14 | 400 | 124 | 31.01 |
| 1.00 | 14 | 390 | 161 | 41.27 | 14 | 454 | 143 | 31.44 |
| 2.00 | 14 | 486 | 181 | 37.18 | 14 | 563 | 163 | 28.88 |
| 4.00 | 14 | 583 | 161 | 27.67 | 14 | 665 | 166 | 24.93 |
| 6.00 | 14 | 636 | 149 | 23.36 | 14 | 671 | 149 | 22.16 |
| 7.00 | 14 | 635 | 160 | 25.20 | 14 | 680 | 144 | 21.22 |
| 8.00 | 14 | 613 | 142 | 23.16 | 14 | 668 | 133 | 19.90 |
| 9.00 | 14 | 599 | 147 | 24.54 | 14 | 644 | 136 | 21.19 |
| 10.00 | 14 | 585 | 144 | 24.64 | 14 | 644 | 143 | 22.29 |
| 11.00 | 14 | 566 | 137 | 24.26 | 14 | 618 | 148 | 23.99 |
| 12.00 | 14 | 577 | 150 | 26.00 | 14 | 607 | 142 | 23.41 |
| 18.00 | 14 | 484 | 140 | 28.98 | 14 | 523 | 141 | 27.02 |
| 24.00 | 14 | 400 | 124 | 31.01 | 14 | 452 | 134 | 29.64 |

Note:
Plasma samples analyzed using a bioanalytical method with a validated range 8.00 to 1500 pg/mL; concentrations reported in pg/mL to 3 significant figures; concentrations below limit of quantification set to zero (0.00 pg/mL) in the data summarization

TABLE 19
Clonidine Concentration-Time Data after Administration
of Sympres 0.1 mg-BID (Treatment B) on Day
6 Excluding Subjects 108 and 110

Treatment B:
Sympres 0.1 mg-BID
Day 6

| Time | | Mean | SD | CV |
|---|---|---|---|---|
| (hr) | n | (pg/mL) | (pg/mL) | (%) |
| 0.00 | 14 | 526 | 148 | 28.12 |
| 1.00 | 14 | 550 | 154 | 28.05 |
| 2.00 | 14 | 613 | 175 | 28.55 |
| 4.00 | 14 | 667 | 161 | 24.11 |
| 5.00 | 14 | 721 | 184 | 25.59 |
| 6.00 | 14 | 667 | 159 | 23.85 |
| 7.00 | 14 | 649 | 149 | 22.89 |
| 8.00 | 14 | 625 | 151 | 24.19 |
| 10.00 | 14 | 580 | 150 | 25.91 |
| 12.00 | 14 | 544 | 139 | 25.46 |
| 13.00 | 14 | 560 | 155 | 27.61 |
| 14.00 | 14 | 603 | 163 | 27.01 |
| 16.00 | 14 | 648 | 152 | 23.51 |
| 17.00 | 14 | 649 | 150 | 23.17 |
| 18.00 | 14 | 644 | 144 | 22.38 |
| 19.00 | 14 | 618 | 138 | 22.34 |
| 20.00 | 14 | 611 | 139 | 22.77 |
| 22.00 | 14 | 575 | 141 | 24.60 |
| 24.00 | 14 | 541 | 138 | 25.47 |

Note:
Plasma samples analyzed using a bioanalytical method with a validated range 8.00 to 1500 pg/mL; concentrations reported in pg/mL to 3 significant figures; concentrations below limit of quantification set to zero (0.00 pg/mL) in the data summarization

TABLE 20
Clonidine Predose Concentration-Time Data after Administration
of CloniDAY 0.2 mg-QD (Treatment A) and Sympres 0.1 mg-BID
(Treatment B) on Days 6 and 7 Excluding Subjects 108 and 110

| | | | | | | Treatment B: | | | |
|---|---|---|---|---|---|---|---|---|---|
| | | Treatment A: | | | | Sympres 0.1 mg-BID | | | |
| | | CloniDAY 0.2 mg-QD | | | | | | SD | |
| | Time | | Mean | SD | CV | | Mean | (pg/ | CV |
| Day | (hr) | n | (pg/mL) | (pg/mL) | (%) | n | (pg/mL) | mL) | (%) |
| 6 | 0 | 14 | 347 | 149 | 42.85 | 14 | 526 | 148 | 28.11 |
| 6 | 12 | 14 | — | — | — | 14 | 545 | 139 | 25.46 |
| 6 | 24 | 14 | 400 | 124 | 31.02 | 14 | 541 | 138 | 25.48 |
| 6 | 48 | 14 | 452 | 134 | 29.65 | 14 | — | — | — |

Note:
Plasma samples analyzed using a bioanalytical method with a validated range 8.00 to 1500 pg/mL; concentrations reported in pg/mL to 3 significant figures; concentrations below limit of quantification set to zero (0.00 pg/mL) in the data summarization

The highest mean plasma clonidine concentrations after administration of CloniDAY 0.2 mg-QD on Days 6 and 7 were 636±149 pg/mL at 6.00 hr and 680±144 pg/mL at 7.00 hr, respectively. The highest mean plasma clonidine concentration after administration of Sympres 0.1 mg-BID on Day 6 was 721±184 pg/mL at 5.00 hr. Quantifiable concentrations of clonidine were observed throughout the 24-hour pharmacokinetic sampling interval for all subjects after each treatment.

### Pharmacokinetic Parameters

Results of the pharmacokinetic and statistical analyses are shown in Tables 21 through 25.

TABLE 21
Pharmacokinetic Parameters of Clonidine Excluding Subjects 108 and 110

| | Treatment A: | | | | Treatment A: | | | | Treatment B: | | | |
|---|---|---|---|---|---|---|---|---|---|---|---|---|
| | CloniDAY 0.2 mg-QD | | | | CloniDAY 0.2 mg-QD | | | | Sympres 0.1 mg-BID | | | |
| | Day 6 | | | | Day 7 | | | | Day 6 | | | |
| Parameter | n | Mean | SD | CV % | n | Mean | SD | CV % | n | Mean | SD | CV % |
| Tₘₐₓ (hr) | 14 | 7.13 | 2.63 | 36.88 | 14 | 6.14 | 2.14 | 34.86 | 14 | 5.01 | 0.39 | 7.84 |
| Tₘₐₓ* | 6.53 [2.00-12.70] | | | | 6.50 [4.00-10.00] | | | | 5.00 [4.00-6.00] | | | |
| Cₘₐₓ (pg/mL) | 14 | 659 | 155 | 23.57 | 14 | 714 | 151 | 21.13 | 14 | 725 | 183 | 25.21 |
| Cₘᵢₙ (pg/mL) | 14 | 327 | 145 | 44.26 | 14 | 389 | 137 | 35.32 | 14 | 507 | 141 | 27.81 |
| AUC_{0-τ} | 14 | 12530 | 3338 | 26.64 | 14 | 13710 | 3257 | 23.75 | 14 | 7410 | 1853 | 25.01 |
| (hr * pg/mL) | | | | | | | | | | | | |
| AUC₀₋₂₄ | 14 | 12530 | 3338 | 26.64 | 14 | 13710 | 3257 | 23.75 | 14 | 14640 | 3536 | 24.16 |
| (hr * pg/mL) | | | | | | | | | | | | |
| AUCₗₐₛₜ | 14 | 12530 | 3338 | 26.64 | 14 | 13720 | 3263 | 23.78 | 14 | 14640 | 3535 | 24.15 |
| (hr * pg/mL) | | | | | | | | | | | | |
| Tₗₐₛₜ (hr) | 14 | 24.00 | 0.00 | 0.00 | 14 | 24.02 | 0.03 | 0.13 | 14 | 24.00 | 0.01 | 0.04 |
| Cₗₐₛₜ (pg/mL) | 14 | 400 | 124 | 31.01 | 14 | 452 | 134 | 29.64 | 14 | 541 | 138 | 25.47 |
| CLₛₛ/F (L/hr) | 14 | 17.06 | 4.568 | 26.77 | 14 | 15.41 | 3.845 | 24.96 | 14 | 14.40 | 4.045 | 28.09 |
| Fluctuation (%) | 14 | 67.21 | 19.48 | 28.99 | 14 | 59.49 | 22.06 | 37.09 | 14 | 35.67 | 7.92 | 22.21 |

Note:
Full precision data used in pharmacokinetic analysis
*Tₘₐₓ presented in hours (hr) as Median [Range]
τ = 24 hours for CloniDAY and 12 hours for Sympres

TABLE 22
Statistical Analysis of the Log-Transformed Systemic Exposure Parameters of
Clonidine Comparing CloniDAY 0.2 mg-QD on Day 7 (Treatment A) to Sympres 0.1 mg-BID
on Day 6 (Treatment B) Excluding Subjects 108 and 110

| Dependent | Geometric Mean^{a} | | Ratio (%)^{b} | 90% CI^{c} | | | ANOVA |
|---|---|---|---|---|---|---|---|
| Variable | Test | Ref | (Test/Ref) | Lower | Upper | Power | CV % |
| ln (Cₘₐₓ) | 698.4608 | 701.9424 | 99.50 | 90.66 | 109.21 | 0.9858 | 13.89 |
| ln (Cₘᵢₙ) | 365.3787 | 488.3563 | 74.82 | 63.52 | 88.13 | 0.7356 | 24.67 |
| ln (AUC₀₋₂₄) | 13346.6341 | 14213.3195 | 93.90 | 84.98 | 103.76 | 0.9760 | 14.90 |

^{a}Geometric Mean for CloniDAY 0.2 mg-QD on Day 7 (Test) and Sympres 0.1 mg-BID on Day 6 (Ref) based on Least Squares Mean of log-transformed parameter values
^{b}Ratio (%) = Geometric Mean (Test)/Geometric Mean (Ref)
^{c}90% Confidence Interval

TABLE 23
Statistical Analysis of the Log-Transformed Systemic Exposure Parameters of
Clonidine Comparing CloniDAY 0.2 mg-QD (Treatment A) Day 6 to Day 7 Excluding Subjects
108 and 110

| | | | | | | | ANOVA | ANOVA |
|---|---|---|---|---|---|---|---|---|
| | | | | | | | CV % | CV % |
| Dependent | Geometric Mean^{a} | | Ratio (%)^{b} | 90% CI^{c} | | | (Intra- | (Inter- |
| Variable | Test | Ref | (Test/Ref) | Lower | Upper | Power | subject) | subject) |
| ln (Cₘₐₓ) | 641.4523 | 698.4608 | 91.84 | 85.13 | 99.07 | 0.9978 | 11.36 | 20.92 |
| ln (Cₘᵢₙ) | 295.8819 | 365.3787 | 80.98 | 72.82 | 90.06 | 0.9634 | 15.97 | 42.14 |
| ln (AUC_{0-τ}) | 12121.2793 | 13346.6341 | 90.82 | 86.19 | 95.69 | 1.0000 | 7.82 | 24.76 |

^{a}Geometric Mean for CloniDAY 0.2 mg-QD on Day 6 (Test) and CloniDAY 0.2 mg-QD on Day 7 (Ref) based on Least Squares Mean of log-transformed parameter values
^{b}Ratio (%) = Geometric Mean (Test)/Geometric Mean (Ref)
^{c}90% Confidence Interval

TABLE 24
Tukey's Test for Comparing Differences in Trough Concentrations
of Clonidine after Administration of CloniDAY 0.2 mg-QD (Treatment
A) on Different Days Excluding Subjects 108 and 110

| Day | 6 | 7 | 8 |
|---|---|---|---|
| 6 | — | 0.5947 | 0.1182 |
| 7 | 0.5947 | — | 0.5601 |
| 8 | 0.1182 | 0.5601 | — |

Note:
p-values are displayed for paired comparisons

TABLE 25
Tukey's Test for Comparing Differences in Trough Concentrations
of Clonidine after Administration of Sympres 0.1 mg-BID (Treatment
B) on Day 6 Excluding Subjects 108 and 110

| Time (hr) | 0 | 12 | 24 |
|---|---|---|---|
| 0 | — | 0.9360 | 0.9579 |
| 12 | 0.9360 | — | 0.9975 |
| 24 | 0.9579 | 0.9975 | — |

Note:
p-values are displayed for paired comparisons

After administration of CloniDAY 0.2 mg-QD and Sympres 0.1 mg-BID, maximum steady state plasma concentrations of clonidine after CloniDAY (Day 7) and Sympres (Day 6) were comparable; maximum steady state plasma concentrations of clonidine were, however, attained earlier after Sympres compared to CloniDAY. Mean Cₘₐₓ values were 659±155 pg/mL after CloniDAY 0.2 mg-QD on Day 6, 714±151 pg/mL after CloniDAY 0.2 mg-QD on Day 7, and 725±183 pg/mL after Sympres 0.1 mg-BID on Day 6 with median [range] Tₘₐₓ values of 6.53 hr [2.00-12.70 hr], 6.50 hr [4.00-10.00 hr], and 5.00 hr [4.00-6.00 hr], respectively. Minimum steady state plasma concentrations of clonidine were lower after CloniDAY 0.2 mg-QD compared to those after Sympres 0.1 mg-BID, where mean Cₘᵢₙ values were 327±145 pg/mL and 389±137 pg/mL after CloniDAY 0.2 mg-QD on Days 6 and 7, respectively, and 507±141 pg/mL on Day 6 after Sympres 0.1 mg-BID.

Overall systemic exposure to clonidine over 24 hours was comparable across the two treatments considered in this study (CloniDAY 0.2 mg-QD and Sympres 0.1 mg-BID) and across study days (CloniDAY 0.2 mg-QD on consecutive days, Days 6 and 7). Mean estimates of AUC₀₋₂₄ were 12530±3338 hr*pg/mL after CloniDAY 0.2 mg-QD on Day 6, 13710±3257 hr*pg/mL after CloniDAY 0.2 mg-QD on Day 7, and 14640±3536 hr*pg/mL on Day 6 after Sympres 0.1 mg-BID. Mean estimates of clearance at steady state (CLₛₛ/F) were also comparable across treatments, 15.41±3.845 L/hr after CloniDAY 0.2 mg-QD (Day 7) and 14.40±4.045 L/hr after Sympres 0.1 mg-BID (Day 6). However, the fluctuation in clonidine concentrations during a dosing interval was approximately 2 times greater after CloniDAY 0.2 mg-QD compared to that for Sympres 0.1 mg-BID; the percent fluctuation was 67.21±19.48% after CloniDAY 0.2 mg-QD on Day 6, 59.49±220.6% after CloniDAY 0.2 mg-QD on Day 7, and 35.67±7.92% after Sympres 0.1 mg-BID on Day 6.

### Statistical Analysis

### Achievement of Steady State

Results from the Tukey's test demonstrated that steady state conditions were attained on Day 6 after administration of both CloniDAY 0.2 mg-QD and Sympres 0.1 mg-BID, where the p-value for comparison of the predose concentrations across dosing days (or intervals) indicated no significant differences (p>0.05).

CloniDAY 0.2 mg-OD on Day 7 vs. Sympres 0.1 mg-BID on Day 6

The steady-state pharmacokinetic parameters for clonidine after a multiple-dosing regimen of CloniDAY 0.2 mg-QD were compared to those after Sympres 0.1 mg-BID. From the ANOVA results, the 90% confidence intervals for comparing Cₘₐₓ and AUC₀₋₂₄ were within the 80% to 125% range, indicating no significant difference in the peak or overall systemic exposure to clonidine between these two treatments at steady state. The geometric mean ratios (90% confidence intervals) were 99.50% (90.66%, 109.21%) for Cₘₐₓ and 93.90% (84.98%, 103.76%) for AUC₀₋₂₄. However, a significant difference was observed for the minimum concentration at steady state, where Cₘᵢₙ after CloniDAY 0.2 mg-QD was approximately 25% lower than that for Sympres 0.1 mg-BID; the geometric mean ratio for Cₘᵢₙ (90% confidence interval) was 74.82% (63.52%, 88.13%).

CloniDAY 0.2 mg-QD on Day 6 vs. CloniDAY 0.2 mg-QD on Day 7

This comparison was made to determine the intersubject and intrasubject variability of the steady-state pharmacokinetic parameters for clonidine during a multiple-dosing regimen of once-daily CloniDAY. From the ANOVA results, the 90% confidence intervals for comparing peak and overall systemic exposure to clonidine across consecutive dosing days at steady state were within the 80% to 125% range. The geometric mean ratios (90% confidence intervals) were 91.84% (85.13%, 99.07%) for Cₘₐₓ and 90.82% (86.19%, 95.69%) for AUC_{0-τ}. The intrasubject and intersubject variability were 11.36% and 20.92%, respectively, for Cₘₐₓ and 7.82% and 24.76%, respectively, for AUC_{0-τ}. The 90% confidence interval for comparing the minimum concentration at steady state was not entirely within the 80% to 125% range. The geometric mean ratio (90% confidence interval) for Cₘᵢₙ was 80.98% (72.82%, 90.06%); the intrasubject and intersubject variability were 15.97% and 42.14%, respectively, for Cₘᵢₙ.

Based on mean estimates of Cₘₐₓ, maximum clonidine plasma concentrations after the administration of CloniDAY 0.2 mg-QD were comparable and occurred later relative to those after administration of Sympres 1×0.1 mg-BID during steady state dosing.

Based on mean estimates of Cₘᵢₙ, minimum clonidine plasma concentrations after the administration of CloniDAY 0.2 mg-QD were lower relative to those after administration of Sympres 0.1 mg-BID during steady state dosing.

Based on mean estimates of AUC₀₋₂₄, overall systemic exposure to clonidine over a 24-hour period after CloniDAY 0.2 mg-QD was comparable to that after Sympres 0.1 mg-BID.

No apparent differences in estimates of clonidine clearance (CLₛₛ/F) were noted between CloniDAY 0.2 mg-QD and Sympres 0.1 mg-BID.

The fluctuation in clonidine concentrations during a dosing interval was approximately 2 times greater after CloniDAY 0.2 mg-QD compared to that for Sympres 0.1 mg-BID; the percent fluctuation was 67.21±19.48% after CloniDAY 0.2 mg-QD on Day 6, 59.49±220.6% after CloniDAY 0.2 mg-QD on Day 7, and 35.67±7.92% after Sympres 0.1 mg-BID on Day 6.

Results from the Tukey's test demonstrated that steady state conditions were attained on. Day 6 after administration of both CloniDAY 0.2 mg-QD and Sympres 0.1 mg-BID, where the p-value for comparison of the predose concentrations across dosing days (or intervals) indicated no significant differences (p>0.05).

From the ANOVA results, the 90% confidence intervals for comparing Cₘₐₓ and AUC₀₋₂₄ after CloniDAY 0.2 mg-QD to those parameters after Sympres 0.1 mg-BID were within the 80% to 125% range, indicating no significant difference in the peak or overall systemic exposure to clonidine between these two treatments at steady state. The geometric mean ratios (90% confidence intervals) were 99.50% (90.66%, 109.21%) for Cₘₐₓ and 93.90% (84.98%, 103.76%) for AUC₀₋₂₄. However, a significant difference was observed for the minimum concentration at steady state, where Cₘᵢₙ after CloniDAY 0.2 mg-QD was approximately 25% lower than that for Sympres 0.1 mg-BID; the geometric mean ratio for Cₘᵢₙ (90% confidence interval) was 74.82% (63.52%, 88.13%).

From the ANOVA results, the 90% confidence intervals for comparing peak and overall systemic exposure to clonidine after CloniDAY 0.2 mg-QD across consecutive days at steady state were within the 80% to 125% range. The geometric mean ratios (90% confidence intervals) were 91.84% (85.13%, 99.07%) for Cₘₐₓ and 90.82% (86.19%, 95.69%) for AUC_{0-τ}. The intrasubject and intersubject variability were 11.36% and 20.92%, respectively, for Cₘₐₓ and 7.82% and 24.76%, respectively, for AUC_{0-τ}. The 90% confidence interval for comparing the minimum concentration at steady state was not entirely within the 80% to 125% range. The geometric mean ratio (90% confidence interval) for Cₘᵢₙ was 80.98% (72.82%, 90.06%); the intrasubject and intersubject variability were 15.97% and 42.14%, respectively, for Cₘᵢₙ.

Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims.

That which is claimed is:

1. An oral dosage form comprising:
(a) an α₂-adrenergic receptor agonist in an amount between 0.001 wt % and 0.5 wt % of said oral dosage form; and
(b) a pharmaceutically acceptable hydrophilic matrix comprising:
(i) at least one hydroxypropyl methylcellulose ether in an amount between 20 wt % and 80 wt % of said oral dosage form;
(ii) at least one of starch, lactose, or dextrose in an amount between 20 wt % and 80 wt % of said oral dosage form; and
(iii) a metal alkyl sulfate;
wherein after administration of said dosage form no more than once about every 24 hours to a subject having a steady state plasma concentration of said α₂-adrenergic receptor agonist, said agonist's plasma concentration peak-to-trough ratio is no greater than about 1.9.

2. The oral dosage form of claim 1, wherein said α₂-adrenergic receptor agonist is clonidine or a pharmaceutically acceptable salt thereof.

3. The oral dosage form of claim 2, wherein said α₂-adrenergic receptor agonist is clonidine hydrochloride.

4. The oral dosage form of claim 2, wherein said amount of clonidine is between about 0.1 wt % to about 0.4 wt % of said oral dosage form.

5. The oral dosage form of claim 1, wherein said amount of α₂-adrenergic receptor agonist is between about 0.1 mg to about 0.7 mg.

6. The oral dosage form of claim 1, wherein said amount of α₂-adrenergic receptor agonist is between about 0.3 mg to about 0.5 mg.

7. The oral dosage form of claim 1, wherein said amount of said metal alkyl sulfate is between about 1 wt % and about 7 wt % of said oral dosage form.

8. The oral dosage form of claim 1, wherein said amount of said metal alkyl sulfate is between about 4 wt % and about 6 wt % of said oral dosage form.

9. The oral dosage form of claim 1, wherein said metal alkyl sulfate is sodium lauryl sulfate.

10. The oral dosage form of claim 1, further comprising:
(c) a metal stearate and/or colloidal silica.

11. The oral dosage form of claim 10, further comprising both a metal stearate and colloidal silica.

12. The oral dosage form of claim 1, wherein said pharmaceutically acceptable hydrophilic matrix comprises:
(i) at least one hydroxypropyl methylcellulose ether in an amount between 20 wt % and 50 wt % of said oral dosage form;
(ii) at least one of starch, lactose, or dextrose in an amount between 40 wt % and 70 wt % of said oral dosage form; and
(iii) a metal alkyl sulfate in an amount between about 1 wt % and about 7 wt %.

13. The oral dosage form of claim 1, wherein said pharmaceutically acceptable hydrophilic matrix comprises:
(i) at least one hydroxypropyl methylcellulose ether in an amount between 30 wt % and 45 wt % of said oral dosage form;
(ii) at least one of starch, lactose, or dextrose in an amount between 50 wt % and 60 wt % of said oral dosage form; and
(iii) a metal alkyl sulfate in an amount between about 4 wt % and about 6 wt %.

14. The oral dosage form of claim 11, comprising:
(a) an α₂-adrenergic receptor agonist in an amount between 0.001 wt % and 0.5 wt % of said oral dosage form; and
(b) a pharmaceutically acceptable hydrophilic matrix comprising:
(i) at least one hydroxypropyl methylcellulose ether in an amount between 30 wt % and 45 wt % of said oral dosage form;
(ii) at least one of starch, lactose, or dextrose in an amount between 50 wt % and 60 wt % of said oral dosage form; and
(iii) a metal alkyl sulfate in an amount between about 4 wt % and about 6 wt %; and
(c) a metal stearate and colloidal silica in an amount between about 0.1 wt % and about 2 wt %.

15. The oral dosage form of claim 11, consisting of:
(a) an α₂-adrenergic receptor agonist in an amount between 0.001 wt % and 0.5 wt % of said oral dosage form; and
(b) a pharmaceutically acceptable hydrophilic matrix comprising:
(i) at least one hydroxypropyl methylcellulose ether in an amount between 30 wt % and 45 wt of said oral dosage form;
(ii) at least one of starch, lactose, or dextrose in an amount between 50 wt % and 60 wt of said oral dosage form; and
(iii) a metal alkyl sulfate in an amount between about 4 wt % and about 6 wt %; and
(c) a metal stearate and colloidal silica in an amount between about 0.1 wt % and about 2 wt %.

16. A method of treating adrenergic dysregulation in a subject in need thereof, comprising administering the oral dosage form of claim 1 to said subject no more than once about every 24 hours, wherein said adrenergic dysregulation is treated.

17. The method of claim 16, wherein said adrenergic dysregulation is manifested in a condition selected from the group consisting of hypertension, atrial fibrillation, congestive heart failure, orthostatic hypotension, postoperative pain, intractable cancer pain, headaches, labor pain, reflex sympathetic dystrophy, akathisia, peripheral neuropathy, neuropathic orofacial pain, diabetic gastroparesis, essential tremor, postepidural shivering, postanesthesia shivering, restless legs syndrome, hypertonicity, hyperkinetic movement disorders, Tourette's syndrome, substance withdrawal, acute anorexia nervosa, attention-deficit/hyperactivity disorder, conduct disorder, bipolar disorder, aggression, narcolepsy, panic disorder, posttraumatic stress disorder, sleep disorders, social phobia, schizophrenia, ulcerative colitis and proctitis, emesis, cyclosporine-induced nephrotoxicity, hyperthyroidism, growth delay in children, excessive sweating, post-menopausal flushing and hot flashes, and any combination thereof.

18. The method of claim 16, wherein said adrenergic dysregulation is manifested in attention-deficit hyperactivity disorder.

19. The method of claim 16, wherein said adrenergic dysregulation is manifested in hypertension.

20. The method of claim 16, wherein said adrenergic dysregulation is manifested in post-menopausal flushing and hot flashes.

21. The method of claim 16, wherein said α₂-adrenergic receptor agonist is clonidine or a pharmaceutically acceptable salt thereof.

22. The method of claim 16, wherein said α₂-adrenergic receptor agonist is clonidine hydrochloride.

23. The method of claim 16, wherein said amount of α₂-adrenergic receptor agonist present in said oral dosage form is between about 0.1 mg to about 0.7 mg.

24. The method of claim 16, wherein said metal alkyl sulfate of said oral dosage form is sodium lauryl sulfate.

25. An oral dosage form comprising an α₂-adrenergic receptor agonist;
wherein after administration of said dosage form no more than once about every 24 hours to a subject having a steady state plasma concentration of said α₂-adrenergic receptor agonist, the AUC per 0.2 mg dose is about 9,000 to about 17,000 h·pg/mL.

26. The oral dosage form of claim 25, wherein after administration of said dosage form no more than once about every 24 hours to a subject having a steady state plasma concentration of said α₂-adrenergic receptor agonist, the Cₘₐₓ is about 500 to about 900 pg/mL after administration of a 0.2 mg dose of the agonist.

27. The oral dosage form of claim 25, wherein after administration of said dosage form no more than once about every 24 hours to a subject having a steady state plasma concentration of said co-adrenergic receptor agonist, the Tₘₐₓ of the agonist after a single dose is about 4 to about 9 hours.

28. The oral dosage form of claim 25, wherein said α₂-adrenergic receptor agonist is clonidine or a pharmaceutically acceptable salt thereof.

29. The oral dosage form of claim 25, wherein said α₂-adrenergic receptor agonist is clonidine hydrochloride.

30. The oral dosage form of claim 25, wherein said AUC is achieved independently of the fasting state or fed state of the subject.

31. A method of treating adrenergic dysregulation in a subject in need thereof, comprising administering the oral dosage form of claim 25 to said subject no more than once about every 24 hours, wherein said adrenergic dysregulation is treated.

32. The method of claim 31, wherein said adrenergic dysregulation is manifested in a condition selected from the group consisting of hypertension, atrial fibrillation, congestive heart failure, orthostatic hypotension, postoperative pain, intractable cancer pain, headaches, labor pain, reflex sympathetic dystrophy, akathisia, peripheral neuropathy, neuropathic orofacial pain, diabetic gastroparesis, essential tremor, postepidural shivering, postanesthesia shivering, restless legs syndrome, hypertonicity, hyperkinetic movement disorders, Tourette's syndrome, substance withdrawal, acute anorexia nervosa, attention-deficit/hyperactivity disorder, conduct disorder, bipolar disorder, aggression, narcolepsy, panic disorder, posttraumatic stress disorder, sleep disorders, social phobia, schizophrenia, ulcerative colitis and proctitis, emesis, cyclosporine-induced nephrotoxicity, hyperthyroidism, growth delay in children, excessive sweating, post-menopausal flushing and hot flashes, and any combination thereof.

33. The method of claim 31, wherein said adrenergic dysregulation is manifested in attention-deficit hyperactivity disorder.

34. The method of claim 31, wherein said adrenergic dysregulation is manifested in hypertension.

35. The method of claim 31, wherein said adrenergic dysregulation is manifested in post-menopausal flushing and hot flashes.

36. The method of claim 31, wherein said α₂-adrenergic receptor agonist is clonidine or a pharmaceutically acceptable salt thereof.

37. The method of claim 31, wherein said α₂-adrenergic receptor agonist is clonidine hydrochloride.

38. An oral dosage form comprising an α₂-adrenergic receptor agonist;
wherein after administration of said dosage form no more than once about every 12 hours to a subject having a steady state plasma concentration of said α₂-adrenergic receptor agonist, the AUC per 0.1 mg dose is about 5,500 to about 9,500 h·pg/mL.

39. The oral dosage form of claim 38, wherein after administration of said dosage form no more than once about every 12 hours to a subject having a steady state plasma concentration of said co-adrenergic receptor agonist, the Cₘₐₓ is about 500 to about 900 pg/mL after administration of a 0.1 mg dose of the agonist.

40. The oral dosage form of claim 38, wherein after administration of said dosage form no more than once about every 12 hours to a subject having a steady state plasma concentration of said α₂-adrenergic receptor agonist, the Tₘₐₓ of the agonist after a single dose is about 4 to about 6 hours.

41. The oral dosage form of claim 38, wherein said α₂-adrenergic receptor agonist is clonidine or a pharmaceutically acceptable salt thereof.

42. The oral dosage form of claim 38, wherein said α₂-adrenergic receptor agonist is clonidine hydrochloride.

43. The oral dosage form of claim 38, wherein said AUC is achieved independently of the fasting state or fed state of the subject.

44. A method of treating adrenergic dysregulation in a subject in need thereof, comprising administering the oral dosage form of claim 39 to said subject no more than once about every 12 hours, wherein said adrenergic dysregulation is treated.

45. The method of claim 44, wherein said adrenergic dysregulation is manifested in a condition selected from the group consisting of hypertension, atrial fibrillation, congestive heart failure, orthostatic hypotension, postoperative pain, intractable cancer pain, headaches, labor pain, reflex sympathetic dystrophy, akathisia, peripheral neuropathy, neuropathic orofacial pain, diabetic gastroparesis, essential tremor, postepidural shivering, postanesthesia shivering, restless legs syndrome, hypertonicity, hyperkinetic movement disorders, Tourette's syndrome, substance withdrawal, acute anorexia nervosa, attention-deficit/hyperactivity disorder, conduct disorder, bipolar disorder, aggression, narcolepsy, panic disorder, posttraumatic stress disorder, sleep disorders, social phobia, schizophrenia, ulcerative colitis and proctitis, emesis, cyclosporine-induced nephrotoxicity, hyperthyroidism, growth delay in children, excessive sweating, post-menopausal flushing and hot flashes, and any combination thereof.

46. The method of claim 44, wherein said adrenergic dysregulation is manifested in attention-deficit hyperactivity disorder.

47. The method of claim 44, wherein said adrenergic dysregulation is manifested in hypertension.

48. The method of claim 44, wherein said adrenergic dysregulation is manifested in post-menopausal flushing and hot flashes.

49. The method of claim 44, wherein said α₂-adrenergic receptor agonist is clonidine or a pharmaceutically acceptable salt thereof.

50. The method of claim 44, wherein said α₂-adrenergic receptor agonist is clonidine hydrochloride.
